(19) United States
(12) Patent Application Publication
(10) Pub. No.: US 2007/0243179 A1
(43) Pub. Date: Oct. 18, 2007

(54) Methods for treating diseases and increasing longevity

(76) Inventor: James P. Elia, Scottsdale, AZ (US)

Correspondence Address: Gerald K. White, Esq.;GERALD K. WHITE & ASSOCIATES, P.C., Suite 835, 205 W. Randolph Street, Chicago, IL, 60606, US

(21) Appl. No.: 11/810,798

(22) Filed: Jun. 7, 2007

Related U.S. Application Data
(63) Continuation of application No. 10/268,833, filed on Oct. 10, 2002.
Application No. 10/268,833 is a continuation-in-part of application No. 10/179,589, filed on Jun. 25, 2002.
Application No. 10/179,589 is a continuation-in-part of application No. 09/064,000, filed on Apr. 21, 1998.

(30) Foreign Application Priority Data
Jun. 30, 1994 (US) PCT/US94/07472
Jun. 17, 2003 (US) PCT/US03/19147

(51) Int. Cl. A61K 35/14

(52) U.S. Cl. 424/93.73

(57) ABSTRACT

Diseases such as cancer, HIV/AIDS, diabetes, infectious diseases, as well as diseases related to the immune and autoimmune systems, are treated through the formation and/or enhancement of the function of organs and suborgans of human patients. An important organ for such purpose is the thymus. Enhancement may be direct or indirect and utilizes energy, enhancement compositions, and/or living organisms to enhance the cells and/or cell products produced by organs and suborgans.

This application is a continuation-in-part of application Ser. No. 10/179,589, filed Jun. 25, 2002, which application is a continuation-in-part of application Ser. No. 09/064,000, filed Apr. 21, 1998.

### BACKGROUND OF THE INVENTION

This invention generally relates to organogenesis and specifically to various methods for growing hard and soft tissue human organs and suborgans. Various techniques for directing and controlling such growth are included in the invention. Also included are methods for treating diseases, increasing longevity, and enhancing the performance of organs, such as a thymus, by enhancing cells and cell products produced by such organ.

The use of genetic materials, such as growth factors, to form buds which subsequently grow into hard and soft tissue organs in human patients is disclosed in U.S. Pat. No. 5,397,235, granted to James P. Elia on Mar. 14, 1995. In addition, U.S. Pat. No. 5,652,225, granted to Jeffrey M. Isner on Jul. 29, 1997; U.S. Pat. No. 6,174,871, granted to H. Kirk Hammond, et al. on Jan. 16, 2001; and U.S. Pat. No. 6,417,205, granted to John Cooke, et al. on Jul. 9, 2002, involve angiogenesis in the human body.

### SUMMARY OF THE INVENTION

Organogenesis methods for the growth of organs, or at least a portion of a desired organ such as a suborgan, in the body of a human patient may be enhanced by inserting or placing genetic material and a physiological nutrient culture in the body. Such genetic material may include a gene and/or a growth factor. Suborgans may include, but are not limited to, a cell, an Islet cell, a group of cells, a neuron, or dermis.

This application also relates to improvements or enhancements of organogenesis methods, such as angiogenesis, by directing and controlling such methods. The various methods involve the formation of organs and suborgans. In vivo and in vitro techniques may be used in the conduct of the invention.

Organogenesis methods for growing at least a portion of a desired organ at a desired site in the body of a human patient may comprise placing a genetic material, capable of causing formation of an organ; directing and controlling organ formation by placing a physiological medium, capable of causing the body to become apoptotic, anti-apoptotic, agonistic, or antagonistic to the induction and formation of the organ; and then growing the organ.

Organ growth may be directed and controlled by placing a genetic material, such as a growth factor, capable of causing organ or suborgan formation and a physiological medium, capable of causing the body to reduce apoptosis, at a desired site of the body. Such procedure permits organ formation and growth to proceed as desired. The above-described placement results in forming a bud in the body from which an organ or suborgan is subsequently grown. Such method illustrates the in vivo aspect of the invention. Organogenesis methods may be further directed and controlled by utilizing physiological mediums, capable of augmenting organogenesis, capable of inhibiting organogenesis, capable of reducing of inflammation, and capable of supercharging cellular environment thereby activating cellular response. Organogenesis inhibitors function to slow, or even cease, organ growth to achieve a desired rate or state of growth.

Organogenesis methods may be enhanced by placing genetic material, capable of forming blood vessels, at a desired site in a human body, and placing a second genetic material, capable of causing a desired organ to form at such site, and then causing the organ to grow in the body.

The invention may also be conducted in in vitro by providing a human cell; contacting such cell with a mixture of a genetic material, for example a growth factor, and a physiological medium; placing such mixture at a desired site in a human body; and thereby forming a bud and subsequently growing at least a portion of an organ thereby.

A variant of the method immediately described above is to permit the cell, genetic material, and physiological medium to form a bud which is then placed into the human body and grown into at least a portion of an organ. A further variant involves permitting growth of at least a portion of an organ in the above-described mixture and then placing newly-grown organ or suborgan into the body at a desired site where further growth may or may not occur.

Another variant of the invention involves placing a genetic material capable of causing blood vessel formation (angiogenesis) at a desired site in the human body, causing blood vessels to form in the body, placing genetic material capable of forming an organ other than the blood vessels at a desired site in the human body, causing a bud and subsequent organ formation at such site. This two-stage organogenesis method prepares the body for organ formation by first creating blood vessels to promote such formation. This method may be utilized with or without a physiological nutrient culture or physiological medium.

Another aspect of the invention involves enhancing the performance of a product(s) of a human organ by treating the organ with energy, an enhancement composition, and/or a living organism to enhance the product, e.g., cell, cell product, and/or other product produced by the organ. The enhancement may be direct or indirect and is performed on a newly formed organ. A thymus is a particularly desirable organ to be enhanced because of its role in the treatment of diseases. The body of the patient may contain a remnant of a pre-existing organ prior to enhancement of the newly formed organ.

Another aspect of the invention involves treating diseases, such as cancer, HIV, AIDS, asthma, acute and chronic infectious diseases, Type 2 diabetes, rheumatoid arthritis, etc., and autoimmune diseases, such as multiple sclerosis, Type 1 diabetes, etc., by placement of a genetic material capable of causing formation of a thymus at a desired site in the body of a human patient; growing the thymus in the body of the patient; and generating a product from the thymus to treat such disease. Typically, such product may comprise T-cells. This enhancement method may be utilized where the patient's immune system has been destroyed, typically by radiation or chemotherapy, prior to utilization of the enhancement method. Physiological mediums and/or physiological nutrient cultures may be utilized as part of the method.

Another aspect of the invention involves treating a human cell with a genetic material and either a physiological medium or a physiological nutrient culture to form a mixture. Such mixture may be: 1) then placed in the body to form a bud and then grow a thymus to treat disease; 2) utilized to form a bud and then such bud placed in a human body and permitted to grow into a thymus to treat disease; 3) utilized to form a bud and a portion of a thymus and then such portion of thymus placed in a human body and permitted to complete growth into a thymus to treat disease; or 4) utilized to form a thymus and then placed such thymus in a human body to treat disease.

Another aspect of the invention comprises treating autoimmune diseases of a human patient. Such method involves eliminating the pre-existing immune system of the patient, placing a genetic material capable of forming a thymus at a desired site in the body, growing the thymus to replace the eliminated, pre-existing immune system, and generating a product, e.g., cell, cell product, or other thymus product to treat the autoimmune disease. This method may further include placing a genetic material capable of causing formation of a treatment organ that generates product(s) that are capable of treating the autoimmune disease. Treatment organs may include the pancreas, Islets of Langerhans, etc. Autoimmune diseases that may be treated by this method include Type 1 diabetes, rheumatoid arthritis, etc.

The above-described methods for treating a disease are also useful for increasing the longevity of a human patient.

The methods of the invention may also be used in combination with a genetic material, such as a growth factor, alone instead of the above-described mixture of genetic material and physiological medium should the user of the method not desire or need to reduce growth inhibition during organ formation.

### DETAILED DESCRIPTION OF THE INVENTION

Growth factors can be utilized to induce the growth of “hard tissue” or bone and “soft tissues” like ectodermal and mesodermal tissues. As used herein, the term growth factor encompasses compositions and living organisms which promote the growth of hard tissue, such as bone, or soft tissue, in the body of a patient. The compositions include organic and inorganic matter. The compositions can be genetically produced or manipulated. The living organisms can be bacteria, viruses, or any other living organism which promote tissue growth. By way of example and not limitation, growth factors can include platelet-derived growth factor (PDGF), epidermal growth factor (EGF), fibroblast growth factor (acidic/basis (FGF a,b), interleukins (IL's), tumor necrosis factor (TNF), transforming growth factor (TGF-B), colony-stimulating factor (CSF), osteopontin (Eta-1 OPN), platelet-derived growth factor (PDGF), interferon (INF), bone morphogenic protein 1 (BMP-1), and insulin growth factor (IGF). Recombinant and non-recombinant growth factors can be utilized as desired. Bacteria or viruses can, when appropriate, be utilized as growth factors. For example, there is a bacterial hydrophilic polypeptide that self-assembles into a nanometer internal diameter pore to build a selective lipid body. Various enzymes can be utilized for the synthesis of peptides which contain amino acids that control three-dimensional protein structure and growth. Growth factors can be applied in gels or other carriers which regulate the rate of release of the growth factors and help maintain the growth factors and the carrier, at a desired location in the body. Time release capsules, granules, or other carriers containing growth factor can be activated by tissue pH, by enzymes, by ultrasound, by electricity, by heat, by selected in vivo chemicals or by any other selected means to release the growth factor. The carrier can be resorbable or non-resorbable. Or, the growth factor itself can be activated by similar means. Either the carrier or the growth factor can mimic extracellular fluid to control cell growth, migration, and function. The growth factor can be administered orally, systemically, in a carrier, by hypodermic needle, through the respiratory tract, or by any other desired method. The growth factor an also be administered into a capsule or other man-made composition or structure placed in the body. While administration of the growth factor is presently usually localized in the patient's body, circumstances may arise where it is advantageous to distribute a growth factor throughout the patient's body in uniform or non-uniform concentrations. An advantage to growth factors is that they can often, especially when in capsule form or in some other containment system, be inserted to a desired site in the body by simply making a small incision and inserting the growth factor. The making of such small incision comprises minor surgery which an often be accomplished on an out-patient basis. The growth factors can be multifactorial and nonspecific.

Examples of some angiogenic growth factors include, but are not limited to:

angiogenin; placental growth factor; angiopoietin-1; platelet-derived endothelial cell growth factor (PD-ECGF); Del-1; platelet-derived growth factor-BB (PDGF-BB); fibroblast growth factor: acidic (aFGF) and basic (bFGF); pleiotrophin (PTN); follistatin; proliferin; granulocyte colony-stimulating factor (G-CSF); transforming growth factor-alpha (TGF-alpha); hepatocyte growth factor (HGF)/scatter factor (SF); transforming growth factor-beta (TGF-beta); interleukin-8 (IL-8); tumor necrosis factor-alpha (TNF-alpha); leptin; vascular endothelial growth factor (VEGF)/vascular permeability factor (VPF); and midkine.

In another embodiment of the invention, genetically produced living material is used to form an implant in the bone of a patient. The DNA structure of a patient is analyzed from a sample of blood or other material extracted from a patient and a biocompatible tooth bud 122 (FIG. 3) is produced. The bud 122 is placed in an opening 123 in the alveolar bone and packing material is placed around or on top of the bud 122. The size of opening 123 can vary as desired. The packing around bud 122 can comprise HAC 124, hydroxyapatite, blood, growth factors, or any other desirable packing material. The bud 122 grows into a full grown tooth in the same manner that tooth buds which are in the jaws of children beneath baby teeth grow into full sized teeth. In a first variation of this embodiment of the invention, analysis of the DNA of the patient is used to identify and select in vitro the genetic material which causes the creation and growth of a tooth bud. This genetic material at least includes a gene or genes, and may include other portions of the DNA. A transcriptional activator is utilized to activate transcription of these tooth bud genes in vitro. An enhancer is used to drive the specific expression of the transcriptional activator. After the enhancer drives the expression of the transcriptional activator, the transcriptional activator transactivates the tooth bud genes. Nutrients and/or other growth factors can be used to sustain and/or promote the creation and growth of, or if appropriate, to cause the differentiation of, a tooth bud after the tooth bud genes are activated. After the tooth bud reaches a desired size, it is transplanted into the jaw bone of a patient. As used herein, the term tooth bud designates a partially grown tooth. Nutrients and/or other growth factors can be used to sustain and promote the growth of, or if appropriate, to cause the differentiation of, the tooth bud after it is transplanted into the jaw of a patient. Instead of tooth bud genes, genes which cause the morphogenesis and further growth of other organs or hard or soft tissue in the body can be identified from the patient's DNA and utilized to grow in vitro organs or tissue for transplant into the body. The organs or tissue can be partially or completely grown at the time of transplant.

In a second variation of the above embodiment of the invention, the structure of the gene or genes which control the growth of a tooth bud in a human being is known, and the genetic material comprises comparable artificially produced genes, or genes harvested from other human beings or animals are transactivated to create and grow a tooth bud. Such artificially produced genes or genes from other animals are transactivated to create and grow a tooth bud in vitro, after which the bud (or other organ or tissue) is transplanted into the body of the patient. The tooth bud grows in a tooth which is comprised of dense, semirigid, porous, calcified skeletal tissue.

In another embodiment of the invention, instead of transplanting a bud 122 into the jaw of a patient, a quantity of genetically produced living material which causes bud 122 to form in the alveolar bone can be placed at a desired position in the alveolar bone such that bud 122 is morphogenetically created in vivo and grows into a full sized tooth. Instead of forming an opening 123, a needle or other means can be used to simply inject the genetically produced living material into a selected location in the alveolar bone. As would be appreciated by those skilled in the art, genetically produced materials can be inserted in the body to cause the body to grow, reproduce, and replace leg bone, facial bone, and any other desired soft and hard tissue in the body. In one variation of this embodiment of the invention, the genetic material is placed at a desired positioning the alveolar bone (by, for example but not by way of limitation, forming an opening 123 to receive the genes or by utilizing a needle to insert the genes at a desired site) to create and grow morphogenetically a tooth bud and, subsequently, a tooth. The genetic material is presently preferably accompanied by a transcriptional activator to turn on the genes' expression, an enhancer to drive the specific expression of the transcriptional activator, and by nutrients and/or other growth factors which promote the in vivo creation and growth of a tooth bud and tooth. The genes can be transcriptionally activated either prior to being inserted or after insertion in the alveolar bone. Instead of tooth bud genes, genes which cause the morphogenetic creation and growth of other organs or other hard or soft tissue in vivo can be identified from the patient's DNA or from another source, and the genetic material can comprise comparable artificially produced genes or genes removed from another animal or otherwise generated. The genetic material is then inserted at the desired locations in a patient's body and utilized to create and grow morphogenetically in vivo organs or other hard or soft tissue. Such genes presently preferably are accompanied by a transcriptional activator to turn on the gene's expression, an enhancer to drive the specific expression of the transcriptional activator, and by nutrients and/or growth factors which promote the creation and growth of a tooth bud and tooth. The genes can be transcriptionally activated prior to or after they are inserted in a patient's body. Any desired substance or means can, as would be appreciated by those of skill in the art, be utilized to cause the activation or initiation of a gene or genes to express themselves by creating and growing morphogenetically an organ or other hard or soft tissue at a desired location or locations(s) in the body of a patient.

The gene or genes used to create and grow morphogenetically a particular organ or other tissue in vivo or in vitro can, if desired and appropriate, be accompanied by or be connected to other genes or DNA material which does not play a part in the growth of the desired organ or other tissue.

In another embodiment of the invention, I provide a method for curing dental disease. The method comprises the step of introducing into the body a substance or form of energy which replaces or alters a gene or genes in the patient's DNA to improve the ability of the patient's to defend against, weaken, or destroy bacteria or viruses which cause dental disease. The replaced or altered genes express themselves in at least some of new cells subsequently produced by the patient's body. For example, the altered or new genes in the patient's DNA may make it more difficult for bacteria, cytokines, or bacterial antigens to penetrate the gum tissue in the mouth of a patient. The particular embodiment of the invention which is preferred is using a chemical substance, heat, electromagnetic energy, or any other means to alter the structure of an existing gene or genes in the patient's DNA or the bacteria's or virus'DNA in vivo, i.e. alters the DNA while the DNA is in the patient's body. This embodiment can be used to improve the body's capability to defend against any disease or illness and is different from current prior art methods of importing new genes which are intended to replace or supersede the original genes existing in the patient's DNA. Morphogenesis or morphogenetics is the origin and evolution of morphological characters and is the growth and differentiation of cells and tissues during development.

Genes express themselves by creating and growing morphogenetically any organ or other hard or soft tissue. Transcriptional activators turn on a gene's expression.

Transcription is the synthesis of messenger RNA (mRNA), the first step in relaying the information contained in DNA. Transcription begins as the interaction between a strand of DNA and the enzyme RNA polymerase. Enzymes can be growth factors. Various enzymes can be utilized in the synthesis of peptides which contain amino acids that control three-dimensional protein structure and growth.

In accordance with the invention, genetic material plus growth factor(s) are implanted directly or indirectly to grow, reproduce, and replace desired soft and hard tissue in the body.

The first step in making an implant is to analyze the DNA. DNA arrays (biochips) and other DNA sequencing methods are known in the art. The genetic material can include a gene or genes and/or other portions of DNA. A transcriptional activator is utilized to activate transcription. The genetic material can be from the patient, can be artificially produced, or can come from other human beings or animals.

Genetic material is well conserved in nature. The Drosophila eyeless gene (ey), the mouse small ey gene (pax-6), and the Aniridia gene in humans are all homologous.

Transgenic animals have attached a promoter (a growth factor) to a specific gene. The resultant initiation of transcription produces a desired protein. For example, human growth hormone can be produced by a farm animal. Promoters are tissue specific. To produce the protein albumin, the gene for albumin is attached to a promoter that is found only in liver tissue. Once the albumin producing promoter—gene pair is inserted into the genome, albumin is produced by future generations.

The initiation of transcription in the fly Drosophila is caused by a transcriptional activator which is obtained from yeast and is called GAL 4. GAL 4 causes tissue specific expression in flies. An upstream gene for eye formation in a fly is ey (eyeless). A growth factor is attached to the ey gene to grow an eye. Two sets of flies are mated to produce a generation of flies having additional eyes.

The first set of flies is genetically engineered to randomly insert GAL 4 into its genome at twenty different locations.

The second set of flies is also genetically manipulated by placing in the eggs of the second set of flies the recombinant eyeless gene and GAL 4 binding sites. The eggs mature to produce flies each having the eyeless gene in every cell in the flies' body.

Genomic engineering of all kinds has created an infinite range of genetic possibilities for implants and growth factors due to DNA cloning and recombinant DNA. Cis position and trans position genes are possible. In addition, annealing techniques allow DNA with DNA, RNA with RNA, or DNA with RNA. Polymerases catalyze the combining of nucleotides to form RNA or DNA. Transcription factors are DNA-binding proteins that control gene activity. Translation is the second step in the relay of genetic information. During translation, the sequence of triplets in mRNA is translated into a corresponding sequence of amino acids to form a polypeptide as the gene product. Termination codons signal the end of translation.

Antisense RNA (or DNA), cDNA's, and expression vector can be genetically manipulated or produced. The term DNA as used herein also includes mitochondrial DNA.

Genomic manipulation can also be based on locating, isolating, attaching, and manipulating single molecules. For example, the process of transcription (as seen through atomic force microscopes) has been halted by the removal of a single nucleoside triphosphate (NTP) that the RNA molecule needed for transcription. Thus, the atomic and subatomic levels are important in genetic engineering.

Genetic engineering can create implants and growth factors which behave in desired manners and produce selected desired results and pathways. As used herein, genetic engineering can create materials that are able to control the flow of matter and/or energy in a deliberate way by spatial, temporal, physicochemical or other physical means alone or in combination.

Desired tissues and organs can also be produced by the process of nucleation.

Genes control structure and function. A gene or bit of genetic material may act as a master control gene which activates thousands of other genes to construct a living organ. Each one of two or more different genes can produce the same organ. For example, in Drosophila, the eye gene and the toy gene both are capable of eye formation.

Since genomic engineering can create a myriad of genetic possibilities, a pathway description of cellular interactions, intracellular and extracellular matrix combinations, and mitogenic or morphogenic stages is impractical.

Complex tissues and organ systems are formed through cellular proliferation and differentiation. This orderly process is regulated by peptide growth factors which are secreted locally and mediate cellular events by triggering cell surface receptors on their target cell(s).

Cells stick together, viruses stick to cells, and white blood cells stick to invading organisms. Optical tweezers developed at Bell Labs in the 1980's can measure and evaluate the “stickiness” of cells and viruses. Sticky cells can be used to attach genetic implants to selected sites. This is, for example, important when placing a soft tissue implant in or on a site of an artery wall. In this manner, an additional heart could be grown from a genetic implant. Once matured to a reasonable state, this new heart can be the body's primary heart and the old heart can be evacuated surgically. Any venous or arterial connections, reconfigurations, or ligations can be surgically attended to. Any other organ can be similarly produced at any desired site in soft or hard tissue.

Genetic implant can form a single precursor area and later split in two. For example, the ET gene causes two eyes to form from a single region.

Multifactorial and nonspecific cells (such as stem cells and germinal cells) can provide the necessary in vivo and in vitro cascade of genetic material once an implanted master control gene's transcription has been activated. Likewise, any host cell, cloned cell, cultured cell, or cell would work. Genetic switches (such as the insect hormone ecdysone) can be used to control genes inserted into humans and animals. These gene switches can also be used in cultured cells or other cells. Gene switches govern whether a gene is on or off making possible precise time of gene activity.

Cellular products and their derivatives can be growth factors. Viral vectors can carry and insert new genes into chromosomes. Growth factors can positively or negatively control genetic transcription. Snippets of DNA with characteristic DNA fingerprints can be used as implant materials. Transcription factor binding sites as well as receptor sites can be genetically engineered and utilized as needed. Receptor sites can also be in the nucleus of cells.

Genetic implants preferably integrate biologically into the host environment.

Murine and human genomes (and perhaps the entire metazoa) are considerably conserved at the nucleic acid and gene linkage levels.

In early tooth germ, bone morphogenic proteins BMP-2 and MPB-4 regulate expression of the homeobox containing genes MSX-1 and MSX-2. These genes, along with the eyeless gene in Drosophila may be considered upstream genes.

The homeobox containing gene MHox regulates the epithelial-mesenchymal interactions required for skeletal organogenesis. The paired-like homeobox gene MHox is required for early events of skeletogenesis in multiple lineages.

The homeobox gene controlling the growth of kidneys has been identified.

Organs, a joint capsule, a ligament, or a ligament with an organ attached, can be grown at any hard or soft tissue site.

Genes express themselves by creating and growing morphogenetically any organ or other hard or soft tissue. Transcriptional activators turn on a gene's expression.

Genes may also play important roles in mechanisms that control the differentiation of structures within and between organs during organogenesis.

Gap junction proteins permit the exchange of regulatory molecules between cells and play important roles during organogenesis.

### EXAMPLE 1

MSX-1 and MSX-2 are the homeobox genes that control the generation and growth of a tooth. A sample of skin tissue is removed from the patient and the MSX-1 and MSX-2 homeobox gene(s) are removed from skin tissue cells. The genes are stored in an appropriate culture medium.

Germinal cells in the process of transcription are obtained from the patient by biopsy or surgical excision. The germinal cells are in hard bone tissue adjacent the apex of the immature forming root of a patient's tooth. These cells are selected because they are actively transcribing root structure and contain active growth and transcription factors which facilitate the formation of the tooth germ. The germinal cells are placed in an appropriate nutrient culture medium outside the patient's body. The homeobox genes MSX-1 and MSX-2 are added to the nutrient culture with the germinal cells. The nutrient culture is maintained at an optimum temperature, which is presently preferably 98.6 degrees F., but can be varied as desired. The homeobox genes MSX-1 and MSX-2 are permitted to bind with transcription factors in germinal cells. After the genes bind with transcription factors, the germinal cells and bound genes are replanted in the patient's body at the tooth site from which the germinal cells were harvested.

### EXAMPLE 2

Example 1 is repeated, except that the homeobox genes are provided with a genetically engineered binding site for attaching to the receptor site on the transcription factor. Similar results are obtained.

### EXAMPLE 3

Example 1 is repeated, except that the germinal cells are obtained from soft periodontal ligament tissue immediately adjacent the apex of the immature forming root of a patient's tooth. These cells are selected because they are actively transcribing root structure and contain active growth and transcription factor which facilitate the formation of the tooth germ.

### EXAMPLE 4

MSX-1 and MSX-2 are the homeobox genes that control the generation and growth of a tooth. A sample of skin tissue is removed from the patient and the MSX-1 and MSX-2 homeobox gene(s) are removed from skin tissue cells. A tooth is removed from the mouth of a patient. The tooth that was removed had an immature root structure. Transcription was occurring at the apex of the tooth that was removed. The homeobox genes MSX-1 and MSX-2 are placed at the apex of socket immediately following the extracting of the tooth. The genes bind with the transcription factor(s) and express themselves to begin the genetic cascade to form early tooth germ. The patient's body completes the formation of the tooth.

### EXAMPLE 5

Example 4 is repeated, except that the homeobox genes are provided with a genetically engineered binding site for attaching to the receptor site on the transcription factor. Similar results are obtained.

### EXAMPLE 6

Example 4 is repeated, except that prior to insertion of the homeobox genes in the tooth socket, tissue on the bottom of the tooth socket is loosened to expose bone cells.

### EXAMPLE 7

Example 4 is repeated, except that after the tooth is pulled, add a transcription factor and energy to activate genes to initiate the formation of tooth germ.

### EXAMPLE 8

Example 7 is repeated, and the transcription factor and energy activate the MSX-2 and MSX-2 genes.

### EXAMPLE 9

Example 1 is repeated, except that BMP-2 and BMP-4 growth factors are obtained by recombinant or natural extraction from bone.

### EXAMPLE 10

MSX-1 and MSX-2 are the homeobox genes that control the generation and growth of a tooth. A sample of skin tissue is removed from the patient and the MSX-1 and MSX-2 homeobox gene(s) are removed from skin tissue cells. The genes are stored in an appropriate nutrient culture medium.

BMP-2 and BMP-4 growth factors are obtained by recombinant or natural extraction from bone.

Living stem cells are harvested from the bone marrow, the blood of the patient, or from cell culture techniques. The stem cells are placed in a nutrient culture medium at 98.6 degrees. The temperature of the culture medium can be varied as desired but ordinarily is between 40 and 102 degrees F.

MSX-1 and MSX-2 transcription factors are obtained which will initiate the expression of the MSX-1 and MSX-2 homeobox genes.

The MSX-1 and MSX-2 transcription factors, BMP-2 and MBP-4 bone morphogenic proteins, and MSX-1 and MSX-2 genes are added to the nutrient culture medium along with the living stem cells.

### EXAMPLE 11

Example 10 is repeated except that the transcription factors bind to a receptor complex in the stem cell nucleus.

### EXAMPLE 12

Example 10 is repeated except that the MSX-1 and MSX-2 transcription factors are not utilized. The transcription of the MSX-1 and MSX-2 homeobox genes is activated by applying an electric spark to the nutrient culture medium.

### EXAMPLE 13

Example 10 is repeated except that the stem cells are starved and the transcription of the MSX-1 and MSX-2 homeobox genes is activated by applying an electric spark to the nutrient culture medium.

### EXAMPLE 14

WT-1 and PAX genes are obtained from a sample of skin tissue removed from the patient. The genes are stored in an appropriate nutrient culture medium. PAX genes produce PAX-2 and other transcription factors.

BMP-7 and other kidney related BMP growth factors are obtained by recombinant or natural extraction from bone.

Living stem cells are harvested from the bone marrow, the blood of the patient, or from cell culture techniques. The stem cells are placed in a nutrient culture medium at 98.6 degrees. The temperature of the culture medium can be varied as desired but ordinarily is between 40 to 102 degrees F.

The WT-1 and PAX genes, and BMP-7 and other kidney BMPS are added to the nutrient culture medium along with the living stem cells.

A primitive kidney germ is produced. The kidney germ is transplanted in the patient's body near a large artery. As the kidney grows, its blood supply will be derived from the artery.

### EXAMPLE 15

The Aniridia gene is obtained from a sample of skin tissue removed from the patient. The gene(s) is stored in an appropriate nutrient culture medium.

Aniridia transcription factor (activates expression of the Aniridia gene) and growth factors (function to help stem cells differentiate during morphogenesis to form an eye) are obtained.

Living stem cells are harvested from the bone marrow, the blood of the patient, or from cell culture techniques. The stem cells are placed in a nutrient culture medium at 98.6 degrees. The temperature of the culture medium can be varied as desired but ordinarily is between 40 to 102 degrees F.

The Aniridia transcription factor and growth factor and the Aniridia gene are added to the nutrient culture medium along with the living stem cells.

A primitive eye germ is produced. The eye germ is transplanted in the patient's body near the optic nerve. As the eye grows, its blood supply will be derived from nearby arteries.

### EXAMPLE 16

The Aniridia gene is obtained from a sample of skin tissue removed from the patient. The gene(s) is stored in an appropriate nutrient culture medium.

Aniridia transcription factor (activates expression of the Aniridia gene) and growth factors (function to help stem cells differentiate during morphogenesis to form an eye) are obtained and added to the nutrient culture medium.

An eye germ develops. A branch of the nearby maxillary artery is translocated to a position adjacent the eye germ to promote the development of the eye germ. The eye germ matures into an eye which receives its blood supply from the maxillary artery.

The term “cell nutrient culture” as used herein can include any or any combination of the following: the extracellular matrix; conventional cell culture nutrients; and/or, a cell nutrient such as a vitamin. As such, the cell nutrient culture can be two-dimensional, three-dimensional, or simply a nutrient, and is useful in promoting the processes of cellular dedifferentiation, redifferentiation, differentiation, growth, and development.

As used herein, the term “physiological nutrient culture” is a selected media(s) to control and direct an event(s) in living host system(s) (i.e., cardiovascular, pulmonary, muscoloskeletal, etc.), organ(s), tissue(s), cell(s). A media is a fluid solution, gel, or quasi-solid solution (mechanical mixture) which supports and directs normal developmental pathways for cell and cell products. An event is one of the sequence of growth, division, cellular aggregation, development of cellular form, development of aggregate cellular form, secretions, etc. which lead to the development of an organ. A physiological nutrient culture can affect macromolecule(s), molecule(s), atom(s), and subatomic particle(s) in said living things. A physiological nutrient culture can include macromolecule(s), molecule(s), atom(s), and subatomic particle(s). A cell nutrient culture is a physiological nutrient culture. A physiological nutrient culture is not necessarily a cell nutrient culture. A physiological nutrient culture promotes cellular survival and cellular proliferation in a desired form(s) or function(s), and promotes differentiation to a selected specific function.

Growth factors control cell growth, division, differentiation, migration, structure, function, and self-assembly. Growth factors include chemical regulators and structural/mechanical regulators. Growth factors, particularly when mimicking the extracellular matrix, exert geometric and nongeometric physical, mechanical, chemical, electrical, and/or structural forces on a cell. They can change a cell's content, shape, form, and/or function. In essence, they can have a kaleidoscopic effect which is very useful in creating and promoting the growth and morphogenesis of irregularly structured cells, tissues, or complex tissues and organs such as neurons, nervous tissue, or the brain. The growth factors can activate and regulate genetic transcription.

The invention utilizes the body as an organ/tissue factory. There may, however, be occasions where the organ/tissue is completely grown ex-vivo before replant or transplant.

Physical examinations can be done on any patient to ascertain applications of the inventions herein described.

Genetic manipulation to any portion of a gene, gene(s), protein, growth factor, or cell(s) whether taken from the patient or from any other source can be done to improve organ or tissue longevity, function, or any other attribute. These materials may be synthesized in any fashion.

The extracellular matrix (ECM) may constantly change as a result of mechanical, endocrine, or genetic factors.

The nutrient package's wall thickness can be two or less nanometers, or it can be any other thickness desired. Its wall can be fabricated from protein or from any other biological or synthetic material desired.

An organ, as used herein, consists of two or more kinds of tissues joined into one structure that has a certain task. For example, the heart is an organ whose job is to circulate blood throughout the body. The heart is made up of connective tissue, muscle tissue, and nervous tissue. Organ systems comprise groups of organs. A major activity in the body is performed by each organ system. For example, the digestive system comprise organs that enable the body to use food. Likewise, the nervous system includes organs the carry signals from one are of the body to another.

Genetic material comprising a portion of a gene, a gene, genes, a gene product (i.e., a composition a gene causes to be produced like, for example, an organ-producing growth factor), growth factor, or an ECM (extracellular matrix) can be used in or on the body to grow an organ to tissue. For example, the vascular epithelial growth factor gene (VEGF) or its growth factor equivalent can be inserted into the body to cause an artery to grow. When insertion of a gene, portion of a gene, gene product, growth factor, or ECM in vivo or ex vivo is referred to herein in connection with any of the implant techniques of the invention, it is understood that a cell nutrient culture(s), physiological nutrient culture(s), carrier (s), enhancer(s), promoter(s), or any other desired auxiliary component(s) can be inserted with the gene or at the same location as the gene, growth factor, ECM, etc.

An artery is an organ from the circulatory system. An artery can be grown in the heart, legs, or other areas by injecting a gene or other genetic material into muscle at a desired site. Size, vascularity, simplicity of access, ease of exploitation, and any other desired factors can be utilized in selecting a desired site. The gene is one of several known VEGF genes which cause the production of vascular endothelial growth factors. Several VEGF genes which produce vascular endothelial growth factors are believed to exist because nature intends for there to be several pathways (i.e., genes) which enable the production of necessary growth factors. The existence of several pathways is believed important because if one of the genes is damaged or inoperative, other similar genes can still orchestrate the production of necessary growth factors. VEGF genes are used by the body to promote blood vessel growth. VEGF genes are assimilated (taken in) by muscle cells. The genes cause the muscle cells to make a VEGF protein which promotes the growth of new arteries. VEGF proteins can be made in a lab and injected into a patient intravenously, intraluminally, or intramuscularly to promote the growth of an artery. Or, the genes (or other genetic material) can be applied with an angioplasty balloon, with the assistance of a vector, or by any other method.

It is not always desirable to grow a completely new organ. Sometimes growing a portion of an organ is desirable. For example, in some heart attacks or strokes, a portion of the heart or brain remains viable and a portion dies. An injection of a gene to form cardiac muscle and/or an injection of a gene to form an artery can be utilized to revive or replace the dead portion of the heart. The dead portion of the heart may (or may not) be used as a matrix while the new muscles and vessels grow. Thus, in this example, a partial new organ is grown in a pre-existing organ. A pacemaker may (or may not) be necessary. A second injection of a gene may (or may not) be necessary to stop cardiac muscle growth once it is completed. Portions of organs throughout the body can similarly be repaired or replaced. It may be necessary to provide gene(s) or growth factor(s) sequentially. For instance, one or more blood vessels are grown by inserting an appropriate gene or other genetic material into a selected area. Second, an appropriate gene or other genetic material is inserted in the selected area to grow a bone or other organ.

The size and shape limitation of the desired structure can come from a containment and boundary contact inhibition phenomenon or by a chemical inhibition.

A variation on the theme of growing a portion of an organ is as follows: a portion of a heart dies. The pericardium is utilized as a scaffold and seeded with cells and/or genes to grow new muscle, and genes (or other genetic material) to grow new arteries. Immediately adjacent the dead cardiac muscle, onto or into the pericardium, the appropriate cells, genes, and/or growth factors (or other genetic material) are placed. Once the new muscle and blood vessels have grown, the function specific tissue can be applied to the damaged portion of the heart and paced, if necessary, to augment cardiac action. If the surgeon desires, the dead muscle can be removed and the new muscle and blood vessels can be surgically rotated into the excised region and secured. This probably can be done endoscopically. In essence, the pericardium is utilized to allow the new muscle wall to grow. The new muscle wall is then transplanted into the damaged heart wall. This procedure utilizes the body as a factor to grow an organ and/or tissue, after which the organ and/or tissue is transplanted to a desired region. On the other hand, the new muscle wall may integrate itself into the old wall and not require transplantation.

It may be advantageous to grow an organ and adjacent tissue. For example, a severe burn victim may lose organs and tissues (skin, blood vessels, fat, muscles, etc.). The gene(s), gene product(s), and/or ECM (or other genetic material) may be assembled utilizing any appropriate delivery vehicle or system. By way of example, and not limitation, four spray cans or other delivery apparatus can be utilized. First, muscle gene in a spray an is applied in a light mist or layer. Then fat, blood vessel, and finally skin gene(s) are applied, each from a separate spray can. Or, possibly, all four components can be admixed in and applied from a single spray can. Carriers, matrixes, isolating layers, and/or form or shape defining products may or may not be used by the operator. All the genes can be in the same spray can or combined with other substances. As can be appreciated by those skilled in the art, any method of inserting the gene(s), growth factors, or ECM into or onto the body can be utilized. Nutrients, analgesics, antiseptics, moisture restoring compositions and methods, and appropriate post-operative dressings can be utilized pursuant to operator discretion on an as-needed basis.

It may be desirable to restore a single function in a multifunctional organ. For example, a pancreas produces digestive enzymes and it produces insulin in the Islets of Langerhans. A practitioner may choose to stimulate only a desired portion. For example, inserting a gene for the creation of more Islets of Langerhans can be utilized to selectively restore an appropriate insulin production level without affecting the production of pancreatic digestive enzymes.

There is a mechanotransduction interplay that occurs from the extracellular matrix (ECM) to and across the cell membrane, through the cell's cytoskeleton, and, to the cell's DNA. Cellular products are produced during this process and the process of morphogenesis is aided by this procedure. It may be possible to rejuvenate an organ by inserting a growth factor (especially a growth factor that can mimic extracellular fluid to control cell growth, division, migration, structure, function, and self-assembly) into or around an organ that no longer operates to optimal capacity or to a desired capacity. For example, in the interplay from the ECM to the DNA as described above, if for any reason the DNA falls into disrepair, cellular fitness and function become altered and a disease state may occur. The organ or tissue no longer functions as well as desired. The insertion of the growth factor into or around the organ may rejuvenate and restore the fitness and function to this organ even though the cellular DNA remains in disrepair. This procedure may, in some cases, allow the cell to repair, restore, change and reverse its DNA damage so that it can replicate normally henceforth. Booster shots of the growth factor may be necessary.

Organs and/or tissues can be formed utilizing the patient's own cells. For example, a skin cell(s) is removed from the intraoral lining of a cheek. The cell is genetically screened to identify DNA damage or other structural and/or functional problems. Any existing prior art genetic screening technique can be utilized. Such methods can utilize lasers, DNA probes, PCR, or any other suitable device. If the cell is damaged, a healthy undamaged cell is, if possible, identified and selected. If a healthy cell cannot be obtained, the damaged cell can be repaired by excision, alkylation, transition, or any other desired method. A growth factor(s) is added to the cell to facilitate dedifferentiation and then redifferentiation and morphogenesis into an organ or function specific tissue. Any machine known in the art can be used to check the genetic fitness of the organ and its stage of morphogenesis. A cell nutrient culture may or may not be utilized depending on the desired functional outcome (i.e., growth of an artery, of pancreatic Islet cells, of a heart, etc.) or other circumstances. Replantation can occur at any appropriate stage of morphogenesis. The foregoing can be repeated without the patient's own cells if universal donor cells such as germinal cells are utilized. Germinal cells do not require a dedifferentiation. They simply differentiate into desired tissues or organs when properly stimulated. Similarly, the DNA utilized in the foreign procedure can come from the patient or from any desired source.

During reimplantation one of the patient's own cells is returned to the patient. During implantation, a cell not originally obtained from the patient is inserted on or in the patient.

In the example above, if germinal cells (and in some cases, stem cells) are utilized, a direct differentiation and morphogenesis into an organ can occur in vivo, ex vivo, or in vitro.

A variant on the above two examples involves inserting a selected gene(s) or portion of a gene into a cell. For example, a cell is removed, analyzed, and repaired if desired or necessary to assure quality (e.g., proper interaction to give structural (protein) or chemical (enzyme) product) and functional outcome (e.g., the production of an organ). A gene(s) or a portion of a gene is secured from the patient cell by sampling or is secured from any other source. The gene is inserted into the cell. A growth factor(s) can be inserted in the cell simultaneously with the gene or at the time preceding or following insertion of the gene. Organ formation occurs and replantation is performed utilizing any acceptable technique. Inserting an appropriate growth factor or other gene product in a cell may, without requiring the insertion of a gene in the cell, trigger the process which causes the cell to grow an organ. Similarly, controlling the ECM contacting a cell can cause mRNA to select and copy a segment of the cell's DNA. This segment of the cell's DNA interacts with one or more components in the cell to produce a growth factor or other gene product which triggers the growth of an organ.

An organ or tissue can be made utilizing pellet, capsule, or other carrier carrying a growth factor, a gene, a growth factor and a gene, or any other desired genetic material. These pellets can include ECM producing compositions or components and can be inserted anywhere in the body. Once inserted in the body, the carriers can be fixed or can be movable; and, they can contain living material, nonliving material, or living and nonliving material. As such, they can be prepackaged pharmaceutical carriers inserted to grow selected tissues and organs. The materials inside the carriers can be from the patient or from any other source. Each carrier can be porous, resorbable, semisolid, gelatinous, or have any other desired physical attribute.

An auxiliary organ or a portion of an auxiliary organ can be grown. For example, a two-chambered auxiliary pump for the heart can be grown. Most heart problems occur on the left side. Augmentation and enlargement of the existing heart can help restore optimal function and help prevent pathological enlargement of a poorly performing section of the heart.

An auxiliary organ can be grown in the body years before the anticipated expiration of the original organ. Genetic or other testing can predict organ failure years in advance allowing an early diagnosis of the future failure of an organ.

Avascular necrosis can be corrected with the insertion of a gene(s) and/or growth factor or other genetic material in the body. For example, avascular necrosis is diagnosed near a joint space. VEGF or BMP genes, or VEFG or BMP growth factors produced by VEFG or BMP genes, respectively, or any other desired genetic based material can be inserted to regrow blood vessels and/or bone. Auxiliary placement apparatus like fixation plates and/or screws, fixing compositions, or any other desired system can be utilized to strengthen or secure tissue. The genes and/or growth factors can be placed adjacent the auxiliary placement apparatus, can be placed in a composition adjacent the auxiliary placement apparatus, can be placed remote from the auxiliary placement apparatus, or can be placed at any other desired location.

Cellular dedifferentiation, differentiation, redifferentiation, and morphogenesis are directed and controlled by growth factors (or their genetic counterparts) controlling cell growth, migration, structure, function, and/or self-assembly. A growth factor (or gene or other genetic material) can be inserted into or onto the body to grow missing limbs or body parts. The insertion of a multifactorial and nonspecific growth factor (or gene) is required. Such a growth factor is pluripotent, senses what body part or other component is missing, and directs adjacent cells to reconstruct the body part along genetically predetermined pathways. The process is not unlike the salamander regrowing a severed tail or limb. Other growth factors may or may not be required.

The insertion of a growth factor (or its gene counterpart) in the body can be utilized to prevent and/or reduce inflammation. Growth factors control cell migration. As such, they can be powerful cell inhibitors to prevent inflammatory cells from migrating into an area. Such an application has major usefulness in the treatment of arthritis or other autoimmune or inflammatory diseases. Thus, a growth factor can be inserted in the body to control cell migration or to perform other functions described herein.

A rotator cuff deficiency often prevents normal sports activities. Ligament dysfunction can prevent jogging. Venous insufficiency can hinder prolonged standing or walking. Such musculoskeletal injuries or deficiencies can be corrected by inserting a gene(s) and/or growth factor(s) or other genetic material into the body to create new tissue and/or organs which replaces or augments existing tissue.

A hybrid organ or other structure can be fabricated genetically to include specific tissues which function as needed. For example, a kidney containing Islets of Langerhans cells can be produced. Such a kidney is useful for a patient with diabetes mellitus and renal failure. Other hybrid structures can be grown according to need.

Gene Trace Systems, In. of Menlo Park, Calif. has developed fully automated DNA sequencing technology that combines DNA probing, sequencing, and sizing reactions with laser-based “time of flight” mass spectrometry. This technology (1) identifies the sequence of base chemicals in a DNA strand in five seconds; (2) permits genetic screening tests that cost as little as a few dollars; and (3) is used for gene discovery and expression, genotyping, and disease diagnosis and identification.

The Biological Microcavity Laser (TBML) analyzes blood and cell samples in minutes. TBML (1) is a kind of “lab-on-a-chip” which utilizes tiny fingers of laser light to image cells which are placed in a small chamber; (2) permits information concerning each cell in a cell sample of millions to be extracted in a few minutes; (3) is a tool for studying cell structure changes and sequencing DNA; (4) can identify the stages of morphogenesis; and (5) is based on a laser device called a VCSEL (vertically-cavity surface-emitting laser). Cells being analyzed with TBML do not have to be killed and stained, as cells normally do, for typical laboratory analysis.

Stem cells associated with the central nervous system differentiate to multiple fates: neurons, astrocytes, and oligodendrocytes. The differentiation of these stem cells is influenced by extracellular signals. For example, platelet-derived growth factor is known to support neuronal differentiation. In contrast, ciliary neurotrophic factor and thyroid hormone T3 act on stem cells to generate astrocytes and oligodendrocytes.

Pax genes are key regulators during organogenesis of kidney, eye, ear, nose, limb, muscle, and vertebral column, and brain.

The extracellular matrix (ECM) is a dense, fibrous network of proteins and sugars forming a complex natural environment surrounding individual cells or groups of cells. Components of the matrix, including proteins such as laminin and fibronectin, bind to specific molecules called integrins on the cell surface. Through these integrins, the matrix sends cells various signals that regulate what genes are active. These signals ultimately influence whether cells proliferate, specialize, migrate, or even eliminate themselves. The ECM has the ability to command cells to use particular, tissue-specific genes. This allows the microenvironment outside of cells to confer tissue specificity. For example, capillary epithelial cells roll up to for normal blood vessels only if grown on the proper matrix molecules.

A gene corresponds to a segment of the DNA that codes for the synthesis of a single polypeptide chain. The definition of a gene product, as used herein, is the polypeptide or ribosomal RNA coded for by a gene, i.e., which a gene causes to be produced. A gene product can include proteins, transcription factor(s), and/or RNA. For example, VEGF is a gene, while VEGF growth factor is a gene product.

Genes, a gene, a portion of a gene, ECM, and/or a nutrient media can be inserted into a cell or groups of cells by direct insertion (for example, an apparatus like a micropipette), with a cell fragment (for example, a plasmid from a bacterium), with a virus vector, liposome, by phagocytosis, with the help of pore-forming substance, electrically, chemically, or by any other desired technique of crossing the cell membrane to reach the nucleus or any other desired site in the cell. A gene(s) can be transferred in the form of naked plasmid DNA. For example, an intramuscular injection can be made of plasmid DNA encoding the secreted angiogenic growth factor such as vascular endothelial growth factor (VEGF).

In accordance with one embodiment of the invention, a gene, growth factor, ECM (or other genetic material) and/or nutrient media is inserted into or onto the body at a specific location to induce and promote the morphogenesis and growth of an organ or desired organ sub-structure at that location. A desired organ sub-structure can comprise a cell, group of cells, neuron, dermis, Islet cells, etc. Also in accordance with the invention, a gene or other genetic material is inserted into or onto a cell or group of cells outside the body to induce and promote morphogenesis and growth of an organ or desired structure. Growth factors can also be utilized in combination with or in place of a gene. The resulting induced organ or other structure is transplanted to a desired location in a patient's body.

Gene products can be inserted in a patient's body to produce an organ or other structure. For example, VEGF growth factor inserted in the body produces an organ, i.e., an artery.

Selected ECM compositions or other environmental factors can induce the morphogenesis of organs or selected organ sub-structures. As used herein, environmental factors include, but are not limited to, compositions which exert physical, mechanical, chemical, electrical, and/or structural forces on living cells.

Another variant of the invention inserts a gene and a growth factor at a selected location or locations in the body of a patient to grow a selected organ or structure. As exemplified by cloning technology, an enucleated ovum is a viable growth factor. Other subunits of a cell also qualify as growth factors. A gene and the extracellular matrix may also be inserted at a selected location or locations in a patient's body to grow an organ. Likewise, a growth factor and the extracellular matrix can be inserted in a patient's body to form an organ.

### EXAMPLE 17

A 36-year old Caucasian male experiences pain in his left leg. A medical examination reveals a damaged one-inch long section of a large artery in his left leg. The examination also reveals that this damaged section of the artery is nearly completely clogged with plaque and that the wall of the artery is weakened. The weakening in the arterial wall makes attempting to clean out the artery risky and also makes it risky to attempt to insert a stent in the artery.

Recombinant cDNA encoded to combine with a cell ribosome to produce the human growth factor VEGF is assembled into a eukaryotic expression plasmid. The recombinant cDNA is from cDNA libraries prepared from HL60 leukemia cells and is known to cause the growth of arteries. The plasmid is maintained at a room temperature of 76 degrees F.

The clones are placed in 1.0 milliliters of a normal saline carrier solution at a room temperature of 76 degrees F. to produce a genetic carrier solution. The genetic carrier solution contains about 250 ug of the cDNA clones. A nutrient culture can, if desired, be utilized in conjunction with or in place of the saline carrier. Each clone is identical. If desired, only a single clone can be inserted in the normal saline carrier solution. The saline carrier solution comprises 0.09% by weight sodium chloride in water. A saline carrier solution is selected because it will not harm the DNA clone.

Two sites are selected for injection of the genetic carrier solution. While the selection of sites can vary as desired, the sites are selected at the lower end (the end nearest the left foot of the patient) of the damaged section of the artery so that the new arterial section grown, can, if necessary, be used to take the place of the damaged section of the artery in the event the damaged section is removed.

The first site is on the exterior wall of the artery on one side of the lower end of the damaged section of the artery. A containment system is placed at the first site.

The second site is inside the wall of the artery on the other side of the lower end of the artery.

The genetic carrier solution is heated to a temperature of 98.6 degrees F. 0.25 milliliters of the genetic carrier solution is injected into the containment system at the first site. 0.25 milliliters of the genetic carrier solution is injected at the second site inside the wall of the artery. Care is taken to slowly inject the genetic carrier solution to avoid entry of the solution into the artery such that blood stream will carry away the cDNA in the solution.

After two weeks, an MRI is taken which shows the patient's leg artery. The MRI reveals new growth at the first and second sites.

After four weeks, another MRI is taken which shows the patient's leg artery. The MRI shows that (1) at the first site, a new artery is growing adjacent the patient's original leg artery, and (2) at the second site, a new section of artery is growing integral with the original artery, i.e., at the second site the new section of artery is lengthening the original artery, much like inserting a new section of hose in a garden hose concentric with the longitudinal axis of the garden hose lengthens the garden hose.

After about eight to twelve weeks, another MRI is taken which shows that the new artery growing adjacent the patient's original artery has grown to a length of about one inch and has integrated itself at each of its ends with the original artery such that blood flows through the new section of artery. The MRI also shows that the new artery at the second site has grown to a length of one-half inch.

In any of the examples of the practice of the invention included herein, cell nutrient culture can be included with the gene, the growth factor, the extracellular matrix, or the environmental factors.

In any of the examples of the practice of the invention included herein, the concept of gene redundancy can be applied. For example, the Examples 1 to 14 concerning a tooth list the genes MSX-1 and MSX-2. These genes differ by only two base pairs. Either gene alone may be sufficient. A further example of redundancy occurs in growth factors. Looking at the Examples 10 to 14, BMP4 or BMP2 alone may be sufficient. Redundancy can also be utilized in connection with transcription factors, extracellular matrices, environmental factors, cell nutrient culture, physiological nutrient cultures, vectors, promoters, etc.

One embodiment of the invention inserts genetic material (gene, growth factor, ECM, etc.) into the body to induce the formation of an organ. Similar inducing materials ex vivo into or onto a living cell in an appropriate physiological nurturing environment will also induce the growth of an organ. The VCSEL laser allows early detection in a living cell of a morphogenic change indicating that organ formation has been initiated. With properly time transplantation, organ growth completes itself.

During the ex vivo application of the invention, a gene and/or growth factor is inserted into a cell or a group of cells; an ECM or environmental factor(s) are placed around and in contact with a cell or group of cells; or, genetic material is inserted into a subunit of a cell to induce organ growth. An example of a subunit of a cell is an enucleated cell or a comparable artificially produced environment. In in vivo or ex vivo embodiments of the invention to induce the growth of an organ, the genes, growth factors, or other genetic material, as well as the environmental factors or cells utilized, can come from any desired source.

### EXAMPLE 18

Genetically produced materials are inserted in the body to cause the body to grow, reproduce, and replace in vivo a clogged artery in the heart. This is an example of site-specific gene expression. A plasmid expression vector containing an enhancer/promoter is utilized to aid in the transfer of the gene into muscle cells. The enhancer is utilized to drive the specific expression of the transcriptional activator. After the enhancer drives the expression of the transcriptional activator, the transcriptional activator transactivates the muscle/artery genes. Saline is used as a carrier. Cardiac muscle can take up naked DNA injection intramuscularly. Injecting plasmid DNA into cardiac (or skeletal) muscle results in expression of the transgene in cardiac myocytes for several weeks or longer.

Readily available off-the-shelf (RAOTS) cDNA clones for recombinant human VEFG165, isolated from cDNA libraries prepared from HL60 leukemia cells, are assembled in a RAOTS expression plasmid utilizing 736 bp CMV promoter/enhancer to drive VEGF expression. Other RAOTS promoters can be utilized to drive VEGF expression for longer periods of time. Other RAOTS recombinant clones of angiogenic growth factors other than VEGF can be utilized, for example, fibroblast growth factor family, endothelial cell growth factor, etc. Downstream from the VEGF cDNA is an SV40 polyadenylation sequence. These fragments occur in the RAOTS pUC118 vector, which includes an Escherichia coli origin of replication and the Beta lactamase gene for ampicillin resistance.

The RAOTS construct is placed into a RAOTS 3 ml syringe with neutral pH physiologic saline at room temperature (or body temperature of about 73 degrees C.). The syringe has a RAOTS 27 gauge needle.

Access to the cardiac muscle is gained by open-heart surgery, endoscopic surgery, direct injection of the needle with incision, or by any other desired means. The cardiac muscle immediately adjacent a clogged artery is slowly injected with the RAOTS construct during a five second time period. Injection is slow to avoid leakage through the external covering of muscle cells. About 0.5 ml to 1.0 ml (milliliter) of fluid is injected containing approximately 500 ug phVEGF165 in saline (N=18). The readily available off-the-shelf cDNA clones cause vascular growth which automatically integrates itself with the cardiac muscle. Anatomic evidence of collateral artery formation is observed by the 30^{th} day following injection to the RAOTS construct. One end of the artery integrates itself in the heart wall to receive blood from the heart. The other end of the artery branches into increasingly smaller blood vessels to distribute blood into the heart muscle. Once the growth of the new artery is completed, the new artery is left in place in the heart wall. Transplantation of the new artery is not required.

Blood flow through the new artery is calculated in a number of ways. For example, Doppler-derived flow can be determined by electromagnetic flowmeters (using, for example, a Doppler Flowmeter sold by Parks Medical Electronic of Aloha, Oreg.) both in vitro and in vivo. Also, RAOTS angiograms or any other readily available commercial devices can be utilized.

VEGF gene expression can be evaluated by readily available off-the-shelf polymerase chain reaction (PCR) techniques.

If controls are desired, the plasmid pGSVLacZ containing a nuclear targeted Beta-galactosidase sequence coupled to the simian virus 40 early promoter can be used. To evaluate efficiency, a promoter-matched reporter plasmid, pCMV Beta (available from Clontech of Palo Alto, Calif.), which encodes Beta-galactosidase under control of CMV promoter/enhancer, can be utilized. Other RAOTS products can be utilized if desired.

### EXAMPLE 19

A patient, a forty-year old African-American female in good health, has been missing tooth number 24 for ten years. The space in her mouth in which her number 24 tooth originally resided is empty. All other teeth except tooth number 24 are present in the patient's mouth. The patient desires a new tooth in the empty “number 24” space in her mouth.

A full thickness mucoperiosteal flap surgery is utilized to expose the bone in the number 24 space. A slight tissue reflection into the number 23 tooth and number 25 tooth areas is carried out to insure adequate working conditions.

A Midwest Quietair handpiece (or other off-the-shelf handpiece) utilizing a #701 XXL bur (Dentsply Midwest of Des Plaines, Ill.) (a #700, #557, #558, etc. bur can be utilized if desired) is used to excavate an implant opening or site in the bone. The implant opening is placed midway between the roots of the number 23 and number 25 teeth. The opening ends at a depth which is about fifteen millimeters and which approximates the depth of the apices of the roots of the number 23 and number 25 teeth. Care is taken not to perforate either the buccal or lingual wall of the bone. In addition, care is taken not to perforate or invade the periodontal ligament space of teeth numbers 23 and 25.

An interrupted drilling technique is utilized to avoid overheating the bone when the #701XXL bur is utilized to form the implant opening. During a drilling sequence, the drill is operated in five-second increments; and the handpiece is permitted to stall. Light pressure and a gentle downward stroke are utilized. The bur is removed from the opening after the handpiece is permitted to stall. This sequence is repeated until an implant opening having the desired depth is created. In the event a standard off-the-shelf implant drill is utilized, the foregoing technique is not utilized and, instead, the manufacturer's recommended drilling technique is followed.

Once the implant opening is created, 0.5 ml of EDTA (ethylene diamine tetra acetic acid) is lavaged to the bottom of the implant opening or site and allowed to set for two minutes. The EDTA solution is then washed off with sterile water. This removes the smear layer which forms when the #701XXL bur is used to form the implant opening.

0.5 cc of propylene glycol alginate solution is mixed with freeze dried MSX-1 matrix proteins. The resultant gel is back loaded into a Luhrlock syringe through an 18-gauge needle. Once loaded, the smaller 27-gauge needle is placed on the syringe to allow the needle to be bent when it is inserted in the implant site in the mouth. The gel loses handling qualities after about two hours and is, therefore, preferably utilized within ten or fifteen minutes after being admixed.

The tip of the 27-gauge needle is placed at the bottom of the implant opening, and 0.25 ml of gel is ejected into the bottom of the implant opening. The needle is slowly removed from the implant opening while, at the same time, the syringe is operated to express additional gel to fill the implant opening from the bottom of the opening to the coronal aspect of the bone surrounding the implant opening. Gum tissue is drawn over the implant opening to close the opening and is sutured in place with Ethicon suture.

Alginate gel begins to be absorbed by the patient's body within 48 hours and binds MSX-1 proteins to bone in or adjacent the implant opening. Within about six (6) months, the formation of a tooth is radiographically confirmed.

### EXAMPLE 20

Example 19 is repeated, except that the MSX-1 alginate matrix proteins are omitted; and in their place, at least one MSX-1 gene, a plasmid, and a promoter/enhancer are mixed with and included in the gel that is loaded into the syringe and injected into the implant opening. Similar results are obtained.

### EXAMPLE 21

Example 19 is repeated, except a 0.09% saline solution is utilized as a carrier instead of the alginate gel. Similar results are obtained.

### EXAMPLE 22

Example 19 is repeated, except a MSX-2 gene is utilized in place of the MSX-1 gene. Similar results are obtained.

### EXAMPLE 23

Example 21 is repeated, except a MSX-2 gene is utilized in place of the MSX-1 gene. Similar results are obtained.

### EXAMPLE 24

Example 20 is repeated, except a PAX-9 gene is utilized in place of the MSX-1 gene. Similar results are obtained.

### EXAMPLE 25

Example 21 is repeated, except a PAX-9 gene is utilized in place of the MSX-1 gene. Similar results are obtained.

### EXAMPLE 26

Example 20 is repeated, except a PAX-9 protein is utilized in place of the MSX-1 gene. Similar results are obtained.

### EXAMPLE 27

Example 21 is repeated, except a PAX-9 protein is utilized in place of the MSX-1 gene. Similar results are obtained.

### EXAMPLE 28

Example 20 is repeated, except at least one MSX-2 gene is included in combination with the MSX-1 gene. Similar results are obtained.

### EXAMPLE 29

Example 21 is repeated, except at least one MSX-2 gene is included in combination with the MSX-1 gene. Similar results are obtained.

### EXAMPLE 30

Example 20 is repeated, except at least one MSX-2 gene is included in combination with the MSX-1 gene, along with BMP2, BMP4, and BMP7 growth factors. Similar results are obtained.

### EXAMPLE 31

Example 21 is repeated, except at least one MSX-2 gene is included in combination with the MSX-1 gene along with BMP2, BMP4, and BMP7 growth factors. Similar results are obtained.

For the development of a tooth in accordance with the invention, an upstream initiator gene(s) and/or growth factor(s) inserted directly in vivo or transplanted into the body at a very early stage of morphogenesis is sufficient for tooth formation. The general approach delineated above for a tooth and an artery is appropriate for any organ or organ system. When an organ is grown ex vivo, other regulator and/or signaling compositions can be utilized in addition to initiator genes (like MSX-1) and/or growth factors. During growth of a tooth, the genetically produced materials noted below can be utilized:

| INITIATION | PROLIFERATION | MORPHOGENESIS |
|---|---|---|
| Bmp2, 4 | Bmp2, 4 | Bmp4 |
| EGF | Dlxl-3 | Collagens |
| FGF8 | EGR1 | Dlxl-3 |
| Lef1 | FGFs | Lef1 |
| Msx1 | Lef1 | Msx1 |
| Msx2 | Msx1 | Msx2 |
| Shh | Msx2 | Notch1-3 |
| | Notch1-3 | Pax9 |
| | Pax9 | RAR |
| | RAR (alpha, beta, omega) | RXR |
| | RXR (alpha, beta, omega) | Tuftelin |
| | Syndecan | |
| | Tenascin | |
| | TGF-beta s | |

The Islets of Langerhans, the initiators, are: Pax-6, Pax-4, and NKX6A. Other factors are the TGF family, Gastrin, IDX-1, PDX-1, INGAP, NeuroD, HNF3beta, IPF-1, helix-loop-helix protein Beta-2, etc.

In accordance with the invention, site preparation prior to the insertion of a gene and/or growth factor into the body can occur at any selected site. For example, examples of site preparation include debridement of a burn wound, the application of EDTA or citric acid to a bone site, or any other desired site preparation. As used herein, genetic material includes a gene(s), a portion of a gene, a growth factor(s), a gene product(s), and/or ECM which individually or collectively function to cause the genesis and growth of an organ.

### EXAMPLE 32

Example 17 is repeated except that the patient is a 24-year old Caucasian male, and the genetic carrier solution is injected into two sites in the right leg of the patient. The first site is on the exterior wall on one side of the right leg artery. The second site is inside the wall of the right leg artery on the other side of the artery. The right leg artery is not blocked and is a normal healthy artery. Similar results are obtained, i.e., a new section of artery grows integral with the original right leg artery, and a new section of artery grows adjacent the original right leg artery.

### EXAMPLE 33

Example 17 is repeated except that VEGF growth factor is utilized in the genetic carrier solution in place of the cDNA. Similar results are obtained.

### EXAMPLE 34

Example 17 is repeated except that the patient is a 32-year old Caucasian female, the cDNA produces a VEGF growth factor which promotes the growth of veins, and the genetic carrier solution is injected into two sites in the right leg of the patient. The first site is on the exterior wall on one side of a large right leg vein. The second site is inside the wall of the right leg vein on the other side of the vein. The right leg vein is not blocked and is a normal healthy vein. Similar results are obtained, i.e., a new section of vein grows integral with the original right leg vein, and a new section of vein grows adjacent the original right leg vein.

### EXAMPLE 35

Example 17 is repeated except that the patient is a 55-year old Caucasian male, and the genetic carrier solution is injected into two sites in the coronary artery of the patient. The first site is on the exterior wall on one side of the artery. The second site is inside the wall of the artery on the other side of the artery. A section of the artery is damaged, is partially blocked, and has a weakened wall. The first and second sites are each below the damaged section of the artery. Similar results are obtained, i.e., a new section of artery grows integral with the original artery, and a new section of artery grows adjacent the original artery. The new section of artery has integrated itself at either end with the original artery so that blood flows through the new section of artery.

An effective means of growing an organ in the body of a human may be to insert into the body a genetic material, such as a growth factor, and a physiological medium. The genetic material, such as a growth factor, has the primary function, of influencing a cell to cause or induce the creation (origin) and formation of an organ. The physiological medium has the secondary function of directing and/or controlling the process of organogenesis which was stimulated or activated by the genetic material. A physiological medium facilitates organogenesis to proceed in an effective manner by overcoming compromising or impairing physiological processes and/or barriers to said organogenesis. A physiological medium can furnish nourishment actively or passively to the organogenesis process. The human body naturally has “checks and balances” which regulate normal (nonpathological) cellular activity. Unfortunately, these checks and balances can be fully or partially opposite in physiological action and, thus, can and do serve as total or partial barriers to organogenesis. To overcome such barriers, it may be necessary and desirable to utilize a physiological medium in conjunction with a genetic material, such as a growth factor, to achieve efficient and complete organogenesis.

An example of the body's system of checks and balances occurs during angiogenesis between the interplay of angiogenic genetic materials and angiogenesis inhibitors. Angiogenesis inhibitors can and do produce proteins which induce apoptosis (programmed cell death). Thus, apoptosis can and does stop the growth of new blood vessels. In Example 36, the use of a physiological medium to regulate and/or stop apoptosis is described.

Physiology is a branch of biology that deals with the functions and activities of life or of living matter (as organs, tissues, or cells) and of the physical and chemical phenomena involved. Physiology also deals with the organic processes and phenomena of an organism or any of its parts of a particular bodily process. Organogenesis is a physiological process. Organogenesis refers to any of the organic processes involved in the origin and development of bodily organs.

Angiogenesis is one of the positive organic processes of the organogenesis process, which can lead ultimately to the formation of the blood vessels of the circulatory system of organs. Angiogenesis inhibitors are one of the negative, or restrictive, organic processes of the organogenesis process, which can prevent new blood vessel growth.

Examples of some angiogenic inhibitors include, but are not limited to: antiangiogenic antithrombin III (aaATIII), 2-methoxyestradiol (2-ME), canstatin, pigment epithelial-derived factor (PEDF), cartilage-derived inhibitor (CDI), placental ribonuclease inhibitor, endostatin (collagen XVIII fragment), plasminogen activator inhibitor, fibronectin fragment, platelet factor-4 (PF4), gro-beta, prolactin 16 kD fragment, heparinases, proliferin-related protein, heparin hexasaccharide fragment, retinoids, human chorionic gonadotropin (hCG), tetrahydrocortisol-S, interferon alpha/beta/gamma, thrombospondin-1, interferon inducible protein (IP-10), transforming growth factor-beta, interleukin-12 (IL-12), tumistatin, kringle 5 (plasminogen fragment), vasculostatin, metalloproteinase inhibitors (TIMPs), vasostatin (caireticulin fragment), and admixtures thereof.

The use of a physiological medium solves the body's problem between any agonistic and/or antagonistic factors such as pro-angiogenic and anti-angiogenic factors. A physiological medium allows organogenesis to proceed where it normally would have ceased or become compromised without the use of said physiological medium.

A physiological medium is a selected medium to direct and control an event in a living host system, organ, tissue, or cell. “Direct” means to dominate and determine any positive, negative, or neutral organic process or phenomenon effecting or involved with organogenesis such that said organogenesis proceeds from creation (origin) to completed organ in time or space in a straightforward manner without substantial deviation, interruption, impairment, impediment, or compromise. “Control” means to substantially regulate, supervise, manipulate, govern, support, augment, supercharge the cellular environment, restrain, guide, manage, activate, deactivate, speed up, slow down, start, stop, influence, rule over, or any act or instance of controlling any positive, negative, or neutral organic process or phenomenon effecting or involved with organogenesis. A physiological medium is used in conjunction with any process involved with human organogenesis.

As used herein, the term “physiological medium” encompasses living matter, non-living matter, or a combination of living and non-living matter from any source. A physiological medium occupies space, has weight, is observable, and possesses energy. In any phase of organogenesis, a physiological medium can exert geometric and/or nongeometric physical, mechanical, chemical, electrical, and/or structural forces to control and direct said organogenesis. A physiological medium may be composed of natural, seminatural, or synthetic materials and may be synthesized in any fashion. A physiological medium can be inserted anywhere in a human body by any means and in any concentration. A physiological medium can be utilized in conjunction with any organogenesis technique or phase of organogenesis, without limitation, including in vivo, ex vivo, and in vitro techniques. A physiological medium can be used with a genetic material, such as a growth factor, to start an organ, to partially grow an organ, or to grow a complete organ. A physiological medium can act intracellularly, extracellularly, intercellularly, or on the cell surface. A physiological medium can regulate precisely, nonprecisely, or in a time-release fashion. A physiological medium can be utilized with any ectodermal, mesodermal, or endodermal tissue. A physiological medium can be utilized for the growth of any hard and/or soft tissue. A physiological medium can be utilized with a genetic material, such as a growth factor, to grow an organ, to grow multiple organs, to grow a specific part(s) of an organ, or to grow an organ to facilitate the repair of an organ (such as growing an artery to repair a heart after a heart attack (a myocardial infarction) or growing an artery to repair a brain after a stroke (cerebrovascular accident). Physiological mediums include organic and inorganic matter, any living organism, genetically produced or manipulated matter, and recombinant and/or non-recombinant matter. Physiological mediums facilitate self-assembly, three-dimensional protein structure and growth, cell migration, cell differentiation, cell structure, and cell function. A physiological medium can be activated or inactivated by thermal energy, electrical, light, sound, or any other form of energy. A physiological medium includes any cell, gene, gene product, intronless gene (minigene), chemokine, cytokine, peptide, or amino acid. A physiological medium encompasses any composition, substance, or matter (living and/or non-living) which acts as a mimetic. A physiological medium includes any ligand and/or its receptor. A physiological medium encompasses any DNA, cDNA, RNA, mRNA, tRNA, and/or EF-Tu protein molecule. A physiological medium can act on any ribosome. A physiological medium can be applied in gels, in saline, by stents, balloons, catheters, or any other carriers. It can be applied locally or systemically. It can be administered orally, systemically, in any carrier, by any needle, parenterally, through the skin, in or on the tongue and/or mouth, through the respiratory tract, or by any other desired method. A physiological medium can be administered in uniform or non-uniform concentrations. It can be injected, inserted through an incision, administered by a skin patch, dispensed by a machine and/or any other type of mechanical device. A physiological medium can be multifactorial and/or non-specific. It can be administered in a capsule, granule, or other man-made composition or structure placed in or on the body. It can be administered by any resorbable or non-resorbable matter. A physiological medium can be activated by certain pH(s), by enzymes, by ultrasound, by selected in vivo, in vitro, or ex vivo chemicals or by any other selected means. A physiological medium encompasses bacteria, plasmids, viruses, or any other living organism. A prion can be utilized in a physiological medium. A physiological medium can work synergistically and/or non-synergistically with any living, non-living, or combination of living and non-living matter. In any phase of organogenesis, a physiological medium can be administered with the genetic material necessary for that selected phase of organogenesis or it can be administered separately. A physiological medium can supercharge any living, non-living, or combination of living and non-living matter. A physiological medium can be used with any genetic material, such as a growth factor, described herein. A physiological medium can be used in conjunction with the growth of any organ subunit, suborgan, or hybrid organ described herein. A physiological medium can supercharge any cellular, extracellular, or intracellular environment. A physiological medium can exhibit cell growth control via retrocrine, autocrine, intracrine, juxtacrine, endocrine, exocrine, and/or paracrine mechanisms. A physiological medium can include any genetic material described herein.

A physiological medium includes any organ- or suborgan-inducing composition or living organism which promotes, induces, or facilitates the formation of any organ or suborgan which then promotes, induces, or facilitates the formation of another organ or suborgan. A physiological medium includes any protein, composition, or living organism that activates, coactivates, or otherwise tricks a cell to “turn on” its genes (express) to promote, induce, or facilitate the formation of an organ or suborgan. A physiological medium includes any composition or living organism that supercharges the promotion, induction, formation, and/or repair of any organ or suborgan. A physiological medium includes any composition, agent, or living organism that is agonistic or antagonistic to the induction and/or formation of an organ or suborgan. A physiological medium includes any composition, agent, or living organism that is anti-apoptotic and/or pro-apoptotic to the induction and/or formation of an organ or suborgan.

An improved method for growing an organ combines a genetic material, such as a growth factor, with a physiological medium. In essence, this is generation two organogenesis. A physiological medium controls and directs processes and phenomena when a genetic material such as a growth factor, is utilized to influence a cell to cause organ formation. For example, an artery is an organ. Angiogenesis would be one of the positive processes involved in the whole organogenesis process of growing an artery. A physiological medium can control and direct the angiogenic process. The angiogenic process involves cell growth, cell proliferation, cell survival, etc.; and, therefore, it is considered a positive process. Angiogenesis inhibitors (whether natural or introduced) would precipitate and/or result in negative, or restrictive, processes to the organogenesis process of growing an artery. It can result in cell death and/or inflammation. This process does not facilitate cell growth, cell proliferation, cell survival, etc; and, therefore, it is considered negative to the organogenesis process. Such angiogenesis (organogenesis) inhibitors could mediate apoptosis, thus stopping the growth of new blood vessels and/or secondarily mediate or inhibit inflammation during and/or following organogenesis. Neither apoptosis nor inflammation is conducive to the growth of an artery. In fact, they would work against a genetic material, such as a growth factor, to cause the growth of an artery. Apoptosis and inflammation may sometimes work synergistically against artery formation. In Example 36, a physiological medium is utilized with a genetic material, such as a growth factor, to overcome the aforementioned negative processes and phenomena.

In Example 37, a physiological medium is utilized with a genetic material, such as a growth factor, to control and direct, and thus augment, a positive process of organogenesis. Again, for purposes of illustration, the organ formed by the genetic material will be an artery. A tumor can cause uncontrolled cell growth. One way a tumor can cause such uncontrolled growth is by making a protein complex that tricks a cell into responding as if the cell were in a state of hypoxia (oxygen deprivation). When a cell is in a state of hypoxia, it turns on genes that induce angiogenesis. Thus, the tumor's protein enslaves cellular machinery to create new blood vessels. Example 37 uses a physiological medium with a genetic material to control and direct the aforementioned phenomenon to augment the positive process of angiogenesis during the organogenesis of an artery.

In Example 38, a physiological medium is utilized in conjunction with a genetic material to grow an organ. Here the physiological medium is utilized to supercharge the cellular environment. As used herein, the term “supercharge” means to charge greatly or excessively. Supercharging the cellular environment can be utilized in any procedure involving a physiological medium used with a genetic material to grow an organ. It is particularly useful for organogenesis procedures where the organ is grown and the body exhibits any state of injury, harm, hurt, damage, impairment, marring, or wounding. In Example 38, the organ is an artery and is grown to repair a heart after a heart attack. Heart muscle is damaged and can be repaired or revived with the genetic material. Supercharging the cellular environment constitutes an improvement over simply using a genetic material to grow an artery and repair the aforesaid conditions.

Any positive organic process in any organogenesis procedure induced by any genetic material, such as a growth factor, can be augmented with a physiological medium. Likewise, any negative organic process in any organogenesis procedure induced by a genetic material can be overcome and/or dominated with a physiological medium. Any kind of supercharging of the cellular environment(s) in any organogenesis procedure induced by a genetic material can be done by utilizing a physiological medium.

Supercharging cellular environment, and thereby activating cellular response to improve organogenesis, may be implemented by including an amino acid in the physiological medium. Suitable amino acids include, but are not limited to, alanine, valine, leucine, isoleucine, proline, methionine, phenylalanine, tryptophan, glycine, serine, threonine, cysteine, asparagine, glutamine, tyrosine, aspartic acid, glutamic acid, lysine, arginine, pyrrolysine, histidine, selenocysteine, and admixtures thereof.

Ligands encompass a group, ion, or molecule coordinated to a central atom or molecule in a complex. Fas ligand (FasL) induces programmed cell death, or apoptosis, in cells expressing its cognate receptor, Fas. Fas is a cell-surface member of the tumor necrosis factor (TNF) receptor superfamily and mediates programmed cell death, or apoptosis, upon engagement by its ligand, FasL. Fas expression is regulated in different cell types by transcription factors that include nuclear factor kB (NF-kB), activator protein 1 (AP-1) and p53. FasL also appears to be regulated by NF-kB and AP-1, as well as by the nuclear factor NF-AT, cMyc, and the interferon regulatory factors 1 and 2 (IRF-1 and IRF-2). Caspace inhibitors can block apoptosis (for example, tri-peptide caspace inhibitor). Inhibitory effects on Fas signaling can occur in the presence of FLICE-inhibitory protein (FLIP) and Fas-associated phosphatase-1 (FAP-1). In addition, the activity of caspaces can be regulated by a family of proteins called inhibitor of apoptosis proteins (IAPs) such as survivin, XIAP, cIAP1, and cIAP2. These proteins can physically interact with, and block, caspace activity. XIAP, cIAP1, and cIAP2 can specifically inhibit caspace-3, -7, and -9, and can inhibit induction of apoptosis in response to diverse stimuli, including FasL. TGF-beta inhibits neutrophil-stimulatory effects of FasL.

Suppressing or inhibiting FasL has a secondary effect. Full-length, membrane-bound FasL is a predominant mediator of inflammatory effects in vivo. This inflammation is secondary to FasL-mediated stimulation of host cells. The process depends on FasL-mediated production of neutrophil chemoattractants by Fas-sensitive cells, rather than on any direct effect of FasL on the neutrophils themselves.

Structurally, Fas has three cystine-rich extracellular domains and an intracellular “death domain” of approximately 80 amino acids, which is required for apoptosis signaling. Blocking of the Fas receptor or of the Fas ligand prevents apoptosis and secondarily inflammation.

### EXAMPLE 36

An example of utilizing a physiological medium in conjunction with a genetic material, such as a growth factor, to control and direct, and thus overcome, two negative organic processes or phenomena effecting or involved with angiogenesis is illustrated below. Controlling and directing the negative processes of apoptosis and inflammation are important to the growth of an organ, such as an artery, and represent an improved method of organogenesis. New blood vessel growth relies upon a balance of proteins that either induce or inhibit new growth of the endothelial cells that form the walls of new blood vessels.

When a genetic material is utilized to grow an artery, endothelial cells, activated by the genetic material, express a cell surface protein receptor called Fas which makes the cells sensitive to angiogenesis inhibitors in their environment. Inhibitors such as thrombospondin-1 (TSP1) or pigment epithelial-derived factor (PEDF), activate the ligand of Fas called FasL. When the cell surface protein FasL fits into the Fas receptor a molecular cascade occurs in the cell that results in cell death, or apoptosis.

However, if a physiological medium containing, for example, a caspace inhibitor is used in conjunction with the genetic material to grow the artery, an improved organogenesis method results. The apoptosis effect precipitated by FasL (which is blocked by the caspace inhibitor) can be prevented. Also prevented is the secondary negative effect of inflammation. Removal of the caspace inhibitor from the physiological medium permits apoptosis, thus stopping arterial growth once a desire state is obtained.

### EXAMPLE 37

A physiological medium is utilized in conjunction with a genetic material to control and direct, and thus augment, positive processes involved in organogenesis. Just as the angiogenic genetic material-angiogenesis inhibitor interplay described in Example 36 leads to the compromising or ceasing of angiogenesis, tumors create a protein complex that enhances angiogenesis. This protein complex, thus, can be utilized in physiological mediums in conjunction with a genetic material as an improved method to grow an organ such as an artery. For example, the activator protein called hypoxia inducing factor (HIF-1) in complex with its coactivator protein called CBP causes genes in the body's cells that induce angiogenesis to turn on. A physiological medium containing the protein complex HIF-1/CBP and/or HIF-1a/CBP when used in conjunction with a genetic material to grow an organ can, in effect, be used to harness the body as a factory and cause the cells to act along with the genetic material to make new blood vessels. In essence, the positive organic processes of organogenesis receive a chorus of support from local in vivo cells as they would be “turned on” by the physiological medium to support the genetic material's primary goal of making an artery. It is one thing to use a genetic material to grow an artery. It is something entirely different to additionally recruit the human body's cells to activate its own natural angiogenic genes to augment the genetic material's ability to grow an artery. This is an example of using a physiological medium to control and direct (above and beyond a genetic material alone) the positive processes of organogenesis.

When organogenesis reaches its desired state, a physiological medium is utilized to stop arterial growth. There is specificity involved in the interaction between HIF-1a and CBP; thus, the addition of a hydroxyl (—OH) group to a single asparagine amino acid within the contact region can completely disrupt the complex. Another technique to cease organogenesis at a desired state is to halt the use of HIF-1/CBP or HIF-1a/CBP in the physiological medium. Any appropriate physiological medium is utilized to negate or limit pathological cellular processes.

### EXAMPLE 38

Sometimes, it is not desirable or necessary to address positive or negative organic processes involved in organogenesis with a physiological medium in conjunction with a genetic material. When neutral processes of organogenesis caused by a genetic material are contemplated, a physiological medium is utilized to effectuate an improved organogenesis method. Neutral processes of organogenesis occur when a genetic material is normally (nonpathologically) controlling cell growth, division, differentiation, migration, structure, function, and self-assembly.

Without altering these neutral processes, a physiological medium is utilized in conjunction with a genetic material to improve organogenesis and/or organ repair by supercharging the cellular environment. For example, after a myocardial infarction, a genetic material is utilized to grow an artery and/or to repair or revive muscle in a heart where part of the heart is dead or compromised. A physiological medium may contain glucose, amino acids, and any antidiabetic (insulin-like) agent. However, the antidiabetic agent forces glucose out of the bloodstream so effectively that hypoglycemia can result. Therefore, supercharging requires monitoring.

Any other nutrient, agent, or supercharging agent may also be included in a physiological medium to nourish and help build and/or rebuild cells. Proteins are built by amino acids, glucose is used by muscle to form glycogen, and an antidiabetic agent actively drives glucose and/or the glucose/amino acid complex out of the bloodstream and into the cellular environment. Thus, artery growth and/or damaged muscle are both actively fed. The physiological medium is used actively to control and direct neutral (or normal) processes and, when used in conjunction with a genetic material, is superior to the effect of a genetic material alone.

The use of a physiological medium with any of the genetic material techniques described in the invention can be utilized. As contemplated herein, a physiological medium is used in conjunction with a genetic material, such as a growth factor, in the process of organogenesis to control, mediate, direct, and/or guide any positive or negative process or to supercharge any process associated with organogenesis.

Supercharging cells is accomplished with the gene or gene product called HOXB4. However, any other supercharging agent can be utilized with a physiological medium to stimulate the production of cells.

A physiological medium can also utilize any of the Bcl-2 family of proteins. Examples of the Bcl-2 family of proteins are Bax and Bak. Within the Bcl-2 family of proteins, some proteins are actively pro-apoptotic while others are anti-apoptotic. A physiological medium can utilize any pro-apoptotic or anti-apoptotic composition or living organism.

A physiological medium can act on or in any cellular organ, such as a mitochondrion.

A cellular response is activated to differing extents by different ligands binding to their receptors. By way of example, and not limitation, receptor superfamilies can include: G protein-linked (or secondary messenger); ligand-gated (or ion channel); tyrosine, kinase, growth factor, and hormone. Agonist ligands cause the full range of activation. Partial agonist ligands can induce some of these responses but not all. Antagonist ligands can disable the signaling of an agonist ligand. The above description of the interplay between receptor superfamilies and their functional (binding) ligands can be utilized to guide any positive, negative, or neutral process of organogenesis.

Cell receptors and their ligands, though important, are just a part of the balance between positive and negative processes that occur during organogenesis. Positive processes or phenomena are needed for continual cellular survival and for organogenesis to continue to completion.

By way of example, and not limitation, positive processes or phenomena in the context of organogenesis controlled and directed by a physiological medium are: cell growth, cell division, cellular aggregation, development of cellular form, development of aggregate cellular form, cell secretion, promotion of cellular survival, promotion of cellular proliferation, promotion of cellular differentiation, protein transport, and signal transduction, etc. By way of example, and not limitation, a physiological medium can utilize or include nutrients which provide metabolic sustenance; antioxidants to fight increased levels of oxidants within the cell; genetic material which acts on a cell and/or another cell (including precursors, inducers, direct inducers, etc.); proteins which enslave cellular machinery; and, anti-apoptotic agents. By way of example, and not limitation, negative processes or phenomena are the opposite of the aforementioned positive processes (for example, cell death, inflammation, cell defects, etc.) and are caused by: increased levels of oxidants within a cell; lack of cellular nutrients; damage to DNA and/or RNA by oxidants and other agents (such as ultraviolet light, x-rays, chemotherapeutic drugs, etc.); failure of genetic materials to influence a cell; lack of proteins to enslave cellular machinery; and pro-apoptotic agents.

Pro-apoptotic agents tumor necrosis factor (alpha and beta) bind to the tumor necrosis factor receptor.

Inhibitors to the caspace superfamily can prevent apoptosis. Members of the caspace superfamily (cystein proteases) promote apoptosis.

Some of the bcl-2 family of proteins promote cell survival (such as: bcl-2, bcl-xL, bag) and some promote cell death (such as bax, bcl-xs, bad, bak).

There are times when it is necessary to utilize a physiological medium to induce organogenesis of one selected organ (for example, an artery); in order to allow a genetic material to subsequently induce successful organogenesis of a second selected organ (for example, a pancreas). In Example 39, if a physiological medium is not used first to induce angiogenesis, the induction and completion of organogenesis of the pancreas is defective.

The use of an artery as an example of organogenesis is important because blood vessels provide inductive signals necessary for the formation of other organs. In the context of organogenesis, if one asks what comes first, the chicken or the egg, the angiogenesis (or vasculogenesis) process starts first and these angiogenic processes then facilitate normal morphogenesis of other organs. Vasculogenic endothelial cells and nascent vessels (buds) are critical for the early morphogenic stages of organogenesis for other organs (other than the blood vessels). Without said activated endothelial cells (or when said activated endothelial cells are inhibited), defects in the other organ's organogenesis processes occur.

The use of a physiological medium to induce the formation of blood vessels serves two purposes in organogenesis: (1) providing necessary inductive signals; and (2) providing necessary ongoing metabolic sustenance for the resulting induced organ. The aforementioned is useful for the genetic material induced formation of a pancreas or a liver. Neither a pancreas bud nor a liver bud (nor any other organ bud) can develop normally without vascular induction. In Example 39, a physiological medium utilizes a genetic material to grow a blood vessel as a means of providing inductive signals to cells which are being influenced by a different genetic material to grow into a different organ (for example, a liver, and/or a pancreas, and/or a suborgan of a pancreas). Thus, the physiological medium will positively effect organogenesis.

A suborgan is a partial or completely functioning unit or portion of an organ. A suborgan is a constituent of an organ serving to perform one particular function (for example, an Islet cell (of the pancreas) that secretes insulin). Another example would be the left ventricle of the heart.

### EXAMPLE 39

A liver can be induced to form by utilizing FGF-1 or FGF-2 and/or BMP and/or Hex.

A pancreas has both an exocrine and an endocrine suborgan component. The exocrine portion of the pancreas makes digestive enzymes. The endocrine portion of the pancreas makes insulin in its Islet cells. Thus, the pancreas is a two-function organ, and each suborgan component is described above. To induce the endocrine portion of the pancreas, insert ngn-3 into endoderm in any region of the body (not necessarily the foregut). For instance, insertion of ngn-3 into the kidney would produce a hybrid organ). This process can be stopped with a physiological medium containing bax, bak, bad, etc.

The exocrine portion of the pancreas would be induced by FGF-7 and/or FGF-10.

Other factors that could be utilized in pancreatogenesis are: Pax-1, Hex-1, PDX-1, and Shh.

Any vasculogenic (where angioblasts differentiate and form primitive tubules) or angiogenic (where primitive tubules branch from pre-existing vessels) genetic material could be inserted conjointly or separately in or with a physiological medium to induce vasculogenesis and/or angiogenesis in order to facilitate the induction from a genetic material of a liver bud, pancreatic bud, or any suborgan portion selected.

An in vitro technique of the above-mentioned organogenesis with a physiological medium is as follows: A cell and an appropriate gene (for example, ngn-3) are utilized in culture and an appropriate gel to induce Islet cell production.

The Islet cells are inserted in vivo with a physiological medium containing an appropriate vasculogenic, angiogenic, and/or genetic material (for example, VEGF).

Any combination of techniques described herein can be utilized with a physiological medium to enhance and/or augment organogenesis and/or suborgan formation. The physiological medium facilitates and/or mediates organogenesis and allows unencumbered organogenesis.

### EXAMPLE 40

The use of a genetic material, such as a growth factor, to induce, promote, and/or facilitate organogenesis in combination with a physiological medium to control and direct said organogenesis is useful for instances where organogenesis forms blood vessels proximate to (in and/or around) internal and external male and/or female sex organs. An example of a male sex organ is the penis. Examples of female sex organs are breasts and ovaries. The newly formed blood vessels facilitate the appearance and function of such organs.

The placement of genetic material, such as a growth factor, and a physiological medium in a human body to cause angiogenesis resulting in blood vessel formation proximate to a male or female sex organ is an aspect of the present invention. New blood vessel formation can improve the function and appearance of human sex organs. Processes such as those capable of augmenting angiogenesis; supercharging cellular environment and thereby activating cellular response; causing the body to become anti-apoptotic to the induction and formation of blood vessels; and causing the body to become agonistic to the induction and formation of blood vessels are useful in benefiting human sex organs. In addition, subsequently inhibiting blood vessel growth by placing an angiogenesis inhibitor in the body once desired blood vessel formation has commenced and occurred is a useful feature to control the above processes.

The use of the above processes and combinations thereof offer the following advantages. The penis is grown and increased in size by the growth of new blood vessels. Increased vascularity may also be used to treat impotency. Such treatment, although it may be performed alone, does not preclude use of erectile disfunction drugs such as Viagra. Likewise, increased vascularity results in increased female breast size, if desired. Increased vascularity in the ovary area is effective in treating infertility.

The methods of the invention are also applicable for accelerating, strengthening, and improving the healing of wounds (whether natural or caused by surgical interventions). Such methods result in an improvement in appearance, including less scarring of the healed wound, as well as reducing inflammation and other post-wound and post-operative complications. The above improvements are the result of the accelerated and enhanced growth of blood vessels at the wound site of a human body. Processes involving the placement of genetic material, such as a growth factor, and a physiological medium to direct and control, and thus assist, the body's healing process are contemplated. Such processes include anti-apoptotic, agonistic, anti-inflammatory, positive, augmenting, and supercharging. Optionally, subsequent treatment with angiogenic inhibitors may be utilized to control or cease blood vessel formation. Activator and/or co-activator proteins may be used as a component of the physiological medium to accelerate healing.

The methods disclosed herein, including supercharging, augmenting, agonistic, antagonistic, apoptotic, anti-apoptotic, positive, or negative, may be practiced individually or in combinations thereof, as appropriate. For example, the organogenesis methods for reducing apoptosis could be utilized with a supercharging method, an inflammation-reducing method, an organ-growth inhibiting method, an organ-growth augmenting method, etc. Likewise, any of the other method(s) could be used with another method(s), as appropriate. It should be further understood that individual methods may be practiced in sequential steps, as appropriate. Moreover, more than one of the same type of method may be used, i.e., two positive methods could be employed together.

As set forth above, organogenesis methods for the growth of organs, or at least a portion of a desired organ such as a suborgan, in the body of a human patient may be controlled and directed by inserting or placing genetic material and a physiological nutrient culture or a physiological medium in the body. Organ growth is benefited because the physiological nutrient culture/physiological medium function to direct and control the organogenesis process. Such techniques may be used for the same organ or for different organs.

### EXAMPLE 41

A nicotine receptor agonist growth factor (such as nicotine) and a peptide which contains amino acids (such as L-arginine, L-lysine) are placed into a human body to form and direct and control the growth of a blood vessel. The use of nicotine receptor agonists such as nicotine is further described in U.S. Pat. No. 6,417,215, entitled “NICOTINE IN THERAPEUTIC ANGIOGENESIS AND VASCULOGENESIS,” granted Jul. 9, 2002 to Cooke, et al.

### EXAMPLE 42

VEGF (which triggers blood vessel development) and a human embryonic stem cell (with human casanova (cas) gene, PTF1p48 and/or PDX-1 genes inserted) are placed into a human body to direct and control the growth of a pancreas bud and subsequent formation of a pancreas. In this example, the blood vessel promotes pancreas differentiation.

Improved effectiveness for treating disease in a human body may be obtained by enhancing a product, such as a cell, a cell product, other organ or suborgan product, etc. (produced by organs and/or suborgans with the organogenesis methods described herein) with forms of energy, enhancement compositions, and living organisms.

“Enhance” as used herein (to describe the aforementioned enhancement for treating disease in the human body) means to promote the product, e.g., cells, cell products, and/or other products of the organ and/or suborgan produced by the organogenesis methods described herein; and/or, to thwart any cell, cell product, organ, substance, and/or condition present in the body that would decrease the effectiveness of said organogenesis cell and/or cell product. The organ and/or suborgan cell and/or cell product (produced by the selected organogenesis method) is enhanced by: increasing their numbers; increasing their strength; increasing their resistance to death; aiding their performance; augmenting their growth, size or intensity; promoting their growth and development, or their well being; increasing their effectiveness and/or ability to work synergistically; producing and/or working with antibodies more effectively and/or efficiently; promoting their structure and/or function, their ability to migrate, differentiate, and/or proliferate; promoting their quality (for example, in HIV-specific CD8+ T-cells, improving their function by making them better able to divide and proliferate, as well as produce higher levels of a molecule called perforin); promoting their ability to palliate; promoting their three-dimensional protein structure and growth; and/or providing an opportunity to work better.

Furthermore, the organ and/or suborgan product, e.g., cell, cell product, and/or other product produced by the organ that was produced by the selected organogenesis method can be enhanced by thwarting any cell, cell product, other organ product, substance and/or condition present in the body that would decrease the effectiveness of said organogenesis cell and/or cell product by: eliminating or substantially eliminating any state, mode, or condition of being (for example, eliminating the immune system); decreasing and/or preventing cells and/or cell products (or by-products) from being produced or rendering the product less effective by other organs, tissues, cells of the body (for example, decreasing the effects of free fatty acids); treating any condition beforehand in an attempt to improve disease treatment by growing an organ (for example, treating breast cancer before growing a thymus); and/or preventing, stopping, or causing anything not to happen that would hamper the treatment of disease by growing a selected organ.

Finally, “enhance” also means to create and/or effectuate any condition anywhere in the body by any means that will improve the treatment, control, prevention, and/or cure of disease by the cells and/or cell products of organs and/or suborgans produced by the organogenesis methods described herein.

Enhancement of cells and/or cell products improves the function of such cells and/or cell products and leads to improved results. Such enhancement can be direct (on the specific cells and/or cell products of the selected organ produced) or indirect (on cells, cell products, tissues, organs, conditions present, or structures elsewhere in the body). The forms of energy, enhancement compositions, and living organisms contemplated are discussed further herein. Such forms of energy, enhancement compositions, and living organisms enhance the treatment of a disease. Forms of energy include, but are not limited to, laser, radiation, heat (thermal), cold, electrical, light, etc. Enhancement compositions include, but are not limited to, colony; stimulating factors; interleukins; cytokines; drugs such as Finasteride (Proscar); chemotherapy agents; adipocytokines, such as tumor necrosis factor-alpha, adiponectin, resistin, etc.; thiazolidinedione class of antidiabetic agents, etc. Living organisms include, but are not limited to, monoclonal antibodies such as Herceptin, etc.

The term “treatment” includes controlling, decreasing, minimizing the invasiveness of care, potentiating and/or functioning with conventional therapy, preventing, and/or curing of any disease or disease condition that is treatable by this invention for a human patient. If it is applicable, relevant, and will produce favorable results in a human patient, then it can be used on any disease. The forms of energy, enhancement compositions, and living organisms are used to augment the organogenesis methods described herein. The forms of energy, enhancement compositions, and living organisms can be used at any time, i.e., before, during or after, the administration of the organogenesis methods, in order to best treat disease. Any administration technique can be used.

As stated above, treatment of diseases may be improved by enhancement of an organ's or suborgan's ability to produce enhanced cells and/or cell products or by creating conditions in the body which, in turn, improve the effectiveness of the medical treatment. Organs suitable for such enhancement include, but are not limited to, the thymus, kidney, pancreas, Islets of Langerhans, liver, etc. Enhancement benefits may be obtained for pre-existing and/or newly formed and grown organs. In the case of a thymus, enhancement of T-cell generation is obtained. In the case of Islets of Langerhans, enhancement of beta cells is obtained. Physiological mediums and physiological nutrient cultures may be employed in combination with the enhancement technique of the present invention.

Conventional forms of disease treatment may, if desired, be used in conjunction with the enhanced method of the invention for treating diseases. Examples of conventional therapy are, but not limited to, surgery, radiation, chemotherapy, bone marrow transplantation, angiogenesis inhibitors, hormone, immunotherapy, gene therapy, drug therapy, etc.

This enhanced method for treating disease in a human body is applicable to, but is not limited to, cancer, diabetes (both Types 1 and 2), autoimmune diseases, acute and chronic infectious diseases, asthma, and resident and non-resident entities in the body with a foreign molecular pattern (including mutations and mutating entities), etc. This enhanced method for treating disease in a human body is also applicable for treatment of thin grafts for burns, and/or for the thin skin resulting from aging and/or sun damage. In this application, the growth of the suborgan dermis is enhanced. Furthermore, the enhanced method for treating disease in a human body is applicable palliatively (mitigatingly) to prolong life and/or to prolong the quality of life by improving the body's defense against disease.

Any clone, antibody molecule, monoclonal antibody, oligonucleotide, gene-trapping technology, or other technology well recognized by those skilled in the medical arts can be utilized in combination with, if desired, this enhanced method for treating disease.

Cancer, as a sporadic disease, accounts for greater than 90 percent of all forms of breast and other cancers, in contrast to heritable forms of cancer which account for a relatively small percentage of the disease. Tumor suppressor genes, such as PTEN, DBC2 (for deleted in breast cancer), etc. are clearly associated with sporadic cancer. Also implicated in cancers are cancer-causing oncogenes, for example, but not limited to, RAS. Cancers associated with the aforementioned can be treated with the techniques and/or combination of techniques described herein.

### EXAMPLE 43

A 56-year old white female patient is diagnosed with an inherited mutation in gene BRCA1 (other examples of inherited mutations in genes associated with breast cancer include, but are not limited to, BRCA2 and p53). She reports a family history of breast cancer, an early onset of menstruation, a late menopause, and she never had children.

She has not yet been diagnosed with cancer. However, she worked her entire career in a plant which exposed her to the industrial chemicals benzene and vinyl chloride.

As an overall wellness measure and also a cancer preventative measure, she wants to not only grow a new thymus but she wants to enhance the cell count in order to better fight any early onset of potential disease. Achievement of the above objectives combine to obtain increased longevity.

The thymus is one of the first of the body tissues to degenerate with age. By the age of 20 years, most humans have less than five percent of original thymic function. Growing a new, fully functional thymus may cause an increase in human longevity (a form of decrease in the aging process).

The thymus is the major organ of the human immune system. It consists of two lobes just below the thyroid gland and above the heart. The health of the thymus is directly proportional to the health of the body. White blood cells originate in the bone marrow. About half of such blood cells enter directly into the bloodstream and tissue fluids (for example, B lymphocytes-B-cells), but the balance pass through the thymus. Examples of T-cells include, but are not limited to: T lymphocytes (T-cells), helper T-cells, memory T-cells, suppressor T-cells, and cytotoxic T-cells. The thymus is central to the immune system because of its primary role in processing white blood cells into T lymphocytes. Thymic lymphocytes perform at least three defensive functions. First, the production and growth of antibodies by other lymphocytes is stimulated. Second, the growth and action of the phagocytes, which surround and engulf foreign matter is stimulated. Finally, thymus lymphocytes recognize and destroy foreign and abnormal tissue. In addition, the thymus releases several hormones such as thymosin and thymic factor, which regulate many other immune functions.

When a cytotoxic T lymphocyte recognizes foreign antigens on the surface of a cell, the cytotoxic T lymphocyte again differentiates, this time into active cells that attack the infected cells directly or into memory cells that continue to circulate in the body. The active cytotoxic T-cells can also release chemicals called lymphokines that draw macrophages. Some (the “killer T-cells”) release cell-killing toxins of their own; some release interferon. Helper T-cells bind to active macrophages and B lymphocytes to produce proteins called interleukins, which stimulate the production of B-cells and cytotoxic T-cells. Suppressor T-cells assist in dampening the activity of the immune system after an infection has been controlled.

The product of white blood cells (including, but not limited to, B-cells and T-cells) can be stimulated by any composition including, but not limited to, colony stimulating factors, interleukins (such as IL-2 and IL-10), etc.

The thymus also produces epithelial cells which form the structure of the organ. T-cells are generated by stem cells.

The 56-year old white female patient is informed preoperatively that one or more thymus organs can be grown in a variety of locations including, but not limited to, the remnant of the thymus, multiple locations of the peritoneum (interperitoneal), multiple locations of the mediastinum, the kidney, the skin, etc. The mediastinum is chosen for one thymus. Using surgical techniques well understood by those skilled in the medical arts, the patient is administered via injection:
A nicotine receptor agonist and ascorbic acid (Vitamin C) to induce a blood supply and to induce (promote) thymic proliferation; and
A human embryonic stem cell or cells with inserted genes (all, one, or any combination thereof):
   Human casanova gene (cas) to prompt differentiation to a precursor cell of endoderm;
   Hoxa 3 and pax 1 genes (transcription factors) to prompt differentiation to thymic epithelial cells;
   Pax 9 gene. Thymopoiesis requires Pax 9 function in thymic epithelial cells;
   Keratinocyte growth factor (KGF or Fgf 7). This gene is a thymus epithelial cell-specific growth factor; and
   Eya 1 gene. This gene is required for the morphogenesis of the thymus.

A thymus is grown in the body of the patient. To enhance the production of B-cells and cytotoxic T-cells of this newly grown thymus, a course of interleukin-2 (IL-2) is administered. To enhance the regulation and production of white blood cells (enabling the body to better combat disease) a course of colony stimulating factors (CSF) is administered. The administration of interleukin-2 and colony stimulating factors can occur one or more times and can be administered over a variable length of time. This enhancement in the treatment of disease (beyond simply improving the organogenesis method alone) results from the administration of IL-2 and CSF which directly enhances the very cell product of the thymus, the T-cell, and the other components of the body the T-cells work with.

### EXAMPLE 44

Example 43 is repeated, except that a nicotine receptor agonist and prostaglandin E1 are used to induce human thymus epithelial stem cells to form a bud and then grow into a thymus.

### EXAMPLE 45

A 65-year old black male patient is diagnosed with prostate cancer. Growing a new thymus is recommended. However, to improve the chances of successful treatment, i.e., that a new thymus will cure his cancer, he must undergo a series of therapies before, during, and after the growth of a new thymus.

Rectal examination and elevated prostate-specific antigen (PSA) tests suggest his prostate condition first be treated with directed and/or shaped beam radiation. He was then administered a course of Finasteride (Proscar). Such drug causes a decrease in dihydrotestosterone (hormone) levels, causing the prostate gland to shrink. Finasteride is administered to the patient for six months. Finally, radioactive chemical “seeds” are implanted into the prostate.

The aforementioned treatments are performed in combination with the growth of a new thymus to render the thymus growth technique more successful.

A thymus is grown.

IL-2 and CSF are administered after a thymus was grown.

### EXAMPLE 46

A 57-year old Asian female patient is diagnosed with breast cancer. Before, during, and after treatments, again, are necessary to enhance the therapeutic effect of growing a new thymus in the body of the patient.

Before growing the thymus, she is treated with monoclonal antibody therapy. Monoclonal antibodies are a highly specific antibody produced in large quantities by clones of an artificially created cell. A monoclonal antibody is a hybrid cell: B-cells are fused with myeloma cells. The B-cell/T-cell interaction, connection, and relevance to the thymus have previously been discussed. In the creation of a monoclonal antibody, a normal B-cell (a type of lymphocyte, or white blood cell) is united with a myeloma cell (a type of cancer). This union results in the formation of cloned hybridomas (hybrid cells) that have the cancer cell's traits of dividing endlessly and the B-cell's ability to produce a specific type of antibody. Monoclonal antibodies are designed to target specific molecules in the body.

The patient is treated with the monoclonal antibody called Herceptin. Herceptin targets cancer cells that overproduce HER-2, the protein implicated in her breast cancer.

After a course of monoclonal antibodies, the patient is grown two thymuses, one in the kidney and one interperitoneal.

After the thymuses are grown, she is administered a course of IL-2 and CSF treatments.

In this example, the monoclonal antibody (living organism) therapy greatly enhanced the new thymus to cure the cancer. Also, the IL-2 and CSF greatly helped stimulate continued T-cell growth in the body.

In some treatments for cancer and other diseases (for example, but not limited to, autoimmune diseases) chemotherapy, radiation, and/or other methods are used to deplete, eliminate, reduce, empty, exhaust, impoverish, bankrupt, remove, eradicate, diminish, and/or dwindle the patient's immune system and/or immune cells. As used herein, the term “alter the immune system and/or immune cells” means any of the aforementioned conditions stated in the preceding sentence. This procedure may be followed because the original immune system is ineffective in fighting the cancer, or the immune system itself caused the disease, i.e., an autoimmune disease).

Examples of autoimmune diseases are Type 1 diabetes, rheumatoid arthritis, multiple sclerosis, systemic lupus erythemastosus (SLE), myasthenia gravis, idoiopathic thrombocytopenia purpura (ITP), scleroderma, alopecia greata, ankylosing spondylitis, antiphospholipid syndrome, autoimmune Addison's disease, autoimmune hemolytic anemia, autoimmune hepatitis, Behcet's disease, bullous pemphigoid, cardiomyopathy, celiac sprue-dermatitis, chronic fatigue immune dysfunction syndrome (DFIDS), chronic inflammatory demyelinating polyneuropathy, Churg-Strauss syndrome, cicatricial pemphigoid, CREST Syndrome, cold agglutinin disease, Crohn's disease, discoid lupus, essential mixed cryoglobulinemia, fibromyalgia-fibromyositis, Graves' disease, Guillain-Barré, Hashimoto's thyroiditis, idiopathic pulmonary fibrosis, Idiopathic IgA nephropathy, insulin dependent diabetes, juvenile arthritis, lichen planus, lupus, Ménière's disease, mixed connective tissue disease, pemphigus vulgaris, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, Raynaud's phenomenon, Reiter's syndrome, rheumatic fever, sarcoidosis, Sjögren's syndrome, stiff-man syndrome, Takayasu arteritis, temporal arteritis/giant cell arteritis, ulcerative colitis, uveitis, vasculitis, vitiligo, Wegener's granulomatosis, etc. In autoimmune diseases, the body considers normal cells to be an infection, and so destroys its own tissues.

The examples above grew a thymus and included other forms of therapy to improve and/or enhance the overall treatment of disease. This combination of therapies may be utilized wherever there is a development of immunodeficiency, where the immune response to infections is very poor. For example, patients having HIV/AIDS and chronic infections, and older patients may benefit from such combination of treatments.

The concepts for the treatment of disease described herein may sometimes involve distinctly different parts of the body interacting complexly with each other; for example, the B-cell/T-cell interaction. The B-cell matures in the bone marrow, and the T-cell matures in the thymus. Antibodies are Y-shaped proteins called immunoglobulins (IG) and are made only by B-cells. Any antibody process can be utilized in this enhanced system to fight, prevent, or cure disease. Examples of antibody processes include, but are not limited to, neutralization, opsonization, and the complement system.

In effect, this concept of enhanced disease treatment is directed to harness and control the complex interactions of body parts on a molecular (biological) level to control, cure, or prevent disease.

### EXAMPLE 47

Sometimes it may be desirable and/or necessary to eliminate the immune system and create a new immune system. If the disease is an autoimmune disease, such as Type 1 diabetes, when the immune system is eliminated, both a new thymus and a new pancreas (or at least the Islets of Langerhans suborgans) may need to be grown in the body of a patient.

A 35-year old white male patient has Type 1 diabetes. The immune system is eliminated with chemotherapy and/or radiation before the new organs are formed.

A new thymus is formed as previously described. A kidney, liver, peritoneum, etc. are growth sites, on which a new thymus may be grown.

A new pancreas (or at least new Islet cell suborgans) is grown with the following procedure:

A human cell or cells, such as a human embryonic stem cell, has the following inserted genes (all, one, or any combination thereof):
Human casanova gene (cas) to differentiate the hESC into endoderm;
PTF1p48 is required for the development of the pancreas, both its exocrine cells (those that secrete digestive enzymes) and its endocrine cells (those that secrete insulin and other hormones);
Sonic hedgehog (Shh) is required for differentiation of endoderm into islet tissue;
PDX-1 gene is required for pancreas development and the expression of beta-cell-specific genes; and
Glucagon-like peptide-1 (GLP1) which stimulates the expression of PDX-1 is required.

To produce the promotion of pancreatic differentiation, a blood vessel is induced using VEGF.

IL-2 and CSF are administered to enhance the thymus' ability to fight disease.

In this example, the treatment of disease is augmented by first administering chemotherapy or radiation. Later, the treatment is further augmented by administering IL-2 and CSF.

### EXAMPLE 48

A 70-year old black female patient has Type 2 diabetes. This disease is characterized by insulin resistance. Insulin resistance is caused by, but not limited to, the release of free fatty acids from adipocytes (fat cells), beta-cell defects (defects of Islet cells), defects in muscle glycogen synthesis, etc.

Many areas of the body, thus, interact to hamper disease treatment. An enhanced method for control of Type 2 diabetes in humans is outlined below:

Grow a new pancreas (or at least new Islet suborgans) at a chosen site.

Modulate the underlying insulin resistance by reducing the effects of the free fatty acids with, for example, but not limited to, adipocytokines, such as tumor necrosis factor-alpha, adiponectin, resistin, etc.

Modulate the insulin resistance in skeletal muscle by improving glucose transport activity and insulin-stimulated muscle glycogen synthesis with the thiazolidinedione class of antidiabetic agents. Thiazolidinediones improve insulin sensitivity and beta-cell (Islet suborgan) insulin secretion.

In this example, disease treatment is enhanced by growing the pancreas (or Islet) and increasing the Islet's insulin secretion (output) and decreasing insulin (cell product of the Islet) resistance.

The range of dosage regimens for Examples 36-48, as described herein are broad. Nanogram to milligram amounts are effective without toxicity. Normally, the genetic material used to induce organogenesis is placed conjointly with the physiological medium, but it can be placed before or after the physiological medium. Continued and/or supplemental administrations of physiological medium can occur.

Genetic material and physiological medium may be mixed together and then placed in the human body or placed in the body separately at approximately the same or different times. When placed in the body separately, the genetic material may be introduced first followed by the physiological medium or the physiological medium may be introduced first to provide a receptive environment for the genetic material.

It should be further understood that the methods of the invention may also be used in combination with a genetic material, such as a growth factor, alone instead of the above-described mixture of genetic material and physiological medium should the user of the method not desire or need to reduce growth inhibition during organ formation. For example, organ formation and growth may be controlled by inhibiting organ growth by placing an organogenesis inhibitor into the body of a human patient once desired growth has occurred.

1-124. (canceled)

125. A method of treating a brain in a human patient having a damaged portion caused by a stroke comprising the steps of placing a growth factor at a selected area of said human patient and forming an artery in said brain thereby causing said damaged portion of said brain to be repaired.

126. The method of claim 125, wherein said growth factor comprises a member selected from the group consisting of cells, cellular products, and derivatives of cellular products.

127. The method of claim 126, wherein said growth factor comprises a cell.

128. The method of claim 127, wherein said cell comprises a stem cell.

129. The method of claim 128, wherein said stem cell is multifactorial and non-specific.

130. A method of treating a brain in a human patient having a dead portion caused by a stroke comprising the steps of placing a growth factor at a selected area of said human patient and forming an artery in said brain thereby causing said dead portion of said brain to be repaired.

131. The method of claim 130, wherein said growth factor comprises a member selected from the group consisting of cells, cellular products, and derivatives of cellular products.

132. The method of claim 131, wherein said growth factor comprises a cell.

133. The method of claim 132, wherein said cell comprises a stem cell.

134. The method of claim 133, wherein said stem cell is multifactorial and non-specific.

135. The method of claim 128, wherein said stem cell is harvested from bone marrow.

136. The method of claim 135, wherein said bone marrow is from said patient.

137. The method of claim 128, wherein said stem cell is harvested from blood.

138. The method of claim 137, wherein said blood is from said patient.

139. The method of claim 133, wherein said stem cell is harvested from bone marrow.

140. The method of claim 139, wherein said bone marrow is from said patient.

141. The method of claim 133, wherein said stem cell is harvested from blood.

142. The method of claim 141, wherein said blood is from said patient.

143. The method of claim 125, wherein said growth factor is locally placed at said selected area.

144. The method of claim 143, wherein said growth factor comprises a stem cell and said stem cell is harvested from bone marrow.

145. The method of claim 144, wherein said bone marrow is from said patient.

146. The method of claim 143, wherein said growth factor comprises a stem cell and said stem cell is harvested from blood.

147. The method of claim 146, wherein said blood is from said patient.

148. The method of claim 130, wherein said growth factor is locally placed at said selected area.

149. The method of claim 148, wherein said growth factor comprises a stem cell and said stem cell is harvested from bone marrow.

150. The method of claim 149, wherein said bone marrow is from said patient.

151. The method of claim 148, wherein said growth factor comprises a stem cell and said stem cell is harvested from blood.

152. The method of claim 151, wherein said blood is from said patient.
